(19) United States
(12) Patent Application Publication
(10) Pub. No.: US 2025/0064364 A1
(43) Pub. Date: Feb. 27, 2025

(54) Blood Collection Adapter and Related Systems and Methods

(71) Applicant: Becton, Dickinson and Company, Franklin Lakes, NJ (US)

(72) Inventors: Qiuxia Hu, Shanghai (CN); Bo Yan, Shanghai (CN); Shuai Wang, Suzhou (CN); Chao Gu, Jiangsu (CN); Yiping Ma, Layton, UT (US); Sivaramakrishnan Balasubramanian, Cary, NC (US); Jonathan Karl Burkholz, Salt Lake City, UT (US); Jordan Ciciliano, Durham, NC (US); Christopher Basciano, Apex, NC (US)

(21) Appl. No.: 18/725,207

(22) PCT Filed: Jan. 10, 2023
(86) PCT No.: PCT/US2023/010472

§ 371 Date: Jun. 28, 2024

Related U.S. Application Data
(60) Provisional application No. 63/300,875, filed on Jan. 19, 2022.

(30) Foreign Application Priority Data
Jan. 11, 2022 (CN) 202210027336.1

(51) Int. Cl. A61B 5/15; A61B 5/153; A61M 39/00; A61M 39/10

(52) U.S. Cl. CPC A61B 5/150992; A61B 5/15003; A61B 5/153; A61M 39/105; A61M 2039/0009; A61M 2039/1072; A61M 2039/1077

(57) ABSTRACT

A catheter system may include a catheter adapter, which may include a distal end, a proximal end, and a lumen extending through the distal end of the catheter adapter and the proximal end of the catheter adapter. The catheter system may include a catheter extending distally from the distal end of the catheter adapter. The catheter system may include a connector coupled to the catheter adapter and comprising a septum. The catheter system may include a blood collection adapter coupled to the connector. The blood collection adapter may be configured to create a fluid path through the connector. The catheter system may include a blood collection set coupled to the blood collection adapter. The blood collection set may include a needle assembly configured to receive a blood collection container.

### CROSS-REFERENCE TO RELATED APPLICATIONS

The present application claims priority to Chinese Application Serial No. 202210027336.1 entitled “Blood Collection Adapter and Related Systems and Methods”, filed Jan. 11, 2022, and U.S. Provisional application Ser. No. 63/300,875, entitled “Blood Collection Adapter and Related Systems and Methods”, filed Jan. 19, 2022, the entire disclosures of which are hereby incorporated by reference in their entirety.

### BACKGROUND OF THE INVENTION

### Field of the Invention

The present disclosure generally relates to a blood collection adapters and related assemblies, systems, and methods for us with a peripheral intravenous catheter (PIVC). The adapters are configured to allow for blood collection via a PIVC both at the time of catheter placement and during catheter indwell.

### Description of Related Art

A catheter is commonly used to infuse fluids into vasculature of a patient. For example, the catheter may be used for infusing normal saline solution, various medicaments, or total parenteral nutrition.

The catheter may include a peripheral intravenous (“IV”) catheter. In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may remove the introducer needle, leaving the catheter in place for future fluid infusion.

Blood draws through peripheral intravenous catheters are not routinely performed, in large part due to a risk of hemolysis of a blood sample withdrawn through the peripheral intravenous catheter. Blood draws are instead frequently performed with blood collection sets, such as, for example, the BD VACUTAINER® ULTRATOUCH™ push button blood collection set, available from Becton, Dickinson & Company of Franklin Lakes, New Jersey. A common problem with using a peripheral intravenous catheter to draw blood from a patient is that as blood is drawn into a syringe or BD VACUTAINER® blood collection tube, red blood cells are in a high shear stress state and susceptible to hemolysis due to a high pressure differential between the vein and the syringe or BD VACUTAINER® blood collection tube. Hemolysis may result in rejection and discard of a blood sample. The high-pressure differential can also result in catheter tip collapse, vein collapse, or other complications.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure generally relates to a blood collection adapters and related assemblies, systems, and methods. In some embodiments, a catheter system may include a catheter adapter, which may include a distal end, a proximal end, and a lumen extending through the distal end of the catheter adapter and the proximal end of the catheter adapter. In some embodiments, the catheter system may include a catheter extending distally from the distal end of the catheter adapter. In some embodiments, the catheter system may include a connector coupled to the catheter adapter. In some embodiments, the connector may include a septum.

In some embodiments, the catheter system may include a blood collection adapter coupled to the connector. In some embodiments, the blood collection adapter may be configured to create a fluid path through the connector. In some embodiments, the catheter system may include a blood collection set coupled to the blood collection adapter. In some embodiments, the blood collection set may include a needle assembly configured to receive a blood collection container.

In some embodiments, the catheter system may include a side port disposed between the distal end of the catheter adapter and the proximal end of the catheter adapter; an extension tube; and an adapter. In some embodiments, a distal end of the extension tube may be integrated with the side port of the catheter adapter and a proximal end of the extension tube may be integrated with the distal end of the adapter. In some embodiments, the adapter may include a distal end and a port. In some embodiments, the connector may be coupled to the port of the adapter. In some embodiments, the adapter may include a Y-adapter.

In some embodiments, the blood collection adapter may include a distal piece and a proximal piece movable distally towards the distal piece. In some embodiments, the proximal piece may be configured to activate the septum and create the fluid path through the connector in response to the proximal piece moving distally towards the distal piece. In some embodiments, the proximal piece of the blood collection adapter may include a cannula configured to penetrate the septum of the connector to create the fluid path through the connector.

In some embodiments, the needle assembly may include a body, a sharp needle extending proximally from the body, and a sheath covering a proximal tip of the sharp needle. In some embodiments, the sharp needle and the cannula may be separately formed and have different gauges. In some embodiments, the sharp needle and the cannula may be separately formed and have the same gauge. In some embodiments, the sharp needle and the cannula may be monolithically formed as a single unit. In some embodiments, the blood collection adapter and the body of the needle assembly may be monolithically formed as a single unit.

In some embodiments, the proximal piece of the blood collection adapter may include a male luer. In some embodiments, the connector may include a needleless connector. In some embodiments, a distal end of the cannula may include a chamfer or a flow diverter.

In some embodiments, the catheter system may include an extension set. In some embodiments, the extension set may include a distal end coupled to the proximal end of the catheter adapter and a proximal end coupled to the connector.

In some embodiments, a blood collection assembly to couple to a catheter assembly may include the needle assembly and the blood collection adapter coupled to a distal end of the needle assembly. In some embodiments, the blood collection adapter may be configured to create the fluid path through a connector. In some embodiments, the blood collection adapter may be directly and removably coupled to the needle assembly. In some embodiments, the blood collection adapter may be monolithically formed as a single unit with the body of the needle assembly.

In some embodiments, a method may include coupling the blood collection assembly to the catheter assembly. In some embodiments, the catheter assembly may include the catheter adapter, which may include the distal end, the proximal end, and the lumen extending through the distal end of the catheter adapter and the proximal end of the catheter adapter. In some embodiments, the catheter assembly may include the catheter and the connector coupled to the catheter adapter. In some embodiments, the connector may include the septum.

In some embodiments, the method may include moving the proximal piece distally towards the distal piece. In some embodiments, the male luer or the cannula may be configured to activate the septum of the connector and create a fluid path through the connector in response to moving the proximal piece distally towards the distal piece. In some embodiments, the method may include collecting blood in a blood collection device coupled to the needle assembly after moving the proximal piece distally towards the distal piece. In some embodiments, the method may include uncoupling the blood collection set, and after removing the needle assembly from the blood collection set, flushing the catheter assembly or infusing fluid through the catheter assembly.

In accordance with another aspect of the present disclosure, a catheter system may be provided including a catheter adapter having a distal end, a proximal end, and a lumen extending through the distal end of the catheter adapter and the proximal end of the catheter adapter. The catheter system may further include a catheter extending distally from the distal end of the catheter adapter and a connector coupled to the catheter adapter and comprising a septum. The catheter system may also include a blood collection adapter coupled to the connector, wherein the blood collection adapter may comprise a cannula configured to penetrate the septum of the connector to create the fluid path through the connector.

In some embodiments, the blood collection adapter may include a body and at least two opposing lever arms coupled to the body via a respective flex point, further wherein the at least two opposing lever arms are configured to releasably couple the blood collection adapter to the connector.

In some embodiments, the blood collection adapter may further include an adapter hub coupled to the body, wherein the adapter hub may include an inner body portion surrounding a portion of the cannula.

In some embodiments, the adapter hub may further include a proximal coupling portion, wherein the proximal coupling portion is configured to couple the blood collection adapter to a luer lock access device.

In some embodiments, the luer lock access device may include a luer lock hub having internal threads capable of engaging the proximal coupling portion of the adapter hub, a needle, and a sheath surrounding the needle.

In some embodiments, the luer lock access device may further include a holder configured to hold a blood collection device.

In some embodiments, the luer lock access device may further include a protective cover at least partially surrounding the needle and sheath.

In some embodiments, the cannula may include a beveled tip.

In some embodiments, the beveled tip of the cannula is configured to extend into only a proximal portion of an interior passage of the connector after the cannula penetrates the septum.

In some embodiments, the cannula may include at least one side hole.

In some embodiments, the at least one side hole of the cannula is configured to extend into only a proximal portion of an interior passage of the connector after the cannula penetrates the septum.

In accordance with another aspect of the present disclosure, a catheter system may be provided including a catheter adapter having a distal end, a proximal end, and a lumen extending through the distal end of the catheter adapter and the proximal end of the catheter adapter. The catheter system may further include a catheter extending distally from the distal end of the catheter adapter and a needleless connector coupled to the catheter adapter and having a septum. The catheter system may also include a blood collection adapter coupled to the needleless connector, wherein the blood collection adapter may include central portion having a blunt tip extending from a distal end thereof and configured to penetrate the septum of the needleless connector to create the fluid path through the needleless connector.

In some embodiments, the blood collection adapter may include at least one side hole disposed adjacent the blunt tip.

In some embodiment, the at least one side hole is configured to extend into only a proximal portion of an interior passage of the needleless connector after the blunt tip penetrates the septum.

In some embodiments, the blood collection adapter may further include an adapter hub coupled to a holder portion, wherein the adapter hub may include an inner body portion surrounding a portion of a cannula.

In some embodiments, the cannula extends between the adapter hub and the blunt tip to form a fluid passage therebetween.

In some embodiments, the adapter hub may further include a proximal coupling portion, wherein the proximal coupling portion is configured to couple the blood collection adapter to a luer lock access device.

In some embodiments, the luer lock access device may include a luer lock hub having internal threads capable of engaging the proximal coupling portion of the adapter hub, a needle, and a sheath surrounding the needle.

In some embodiments, the luer lock access device may further include a holder configured to hold a blood collection device.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of an example catheter system that includes hemolysis protection, illustrating an example blood collection adapter in a proximal position, according to some embodiments;

FIG. 1B is an upper perspective view of the catheter system, illustrating the blood collection adapter in a distal position, according to some embodiments;

FIG. 1C is a cross-sectional view of a portion of the catheter system, illustrating the blood collection adapter in the proximal position, according to some embodiments;

FIG. 1D is an exploded view of the portion of the catheter system, according to some embodiments;

FIG. 1E is a cross-sectional view of the portion of the catheter system, illustrating the blood collection adapter in the distal position, according to some embodiments;

FIG. 1F is a cross-sectional view of the portion of the catheter system, illustrating the blood collection adapter, according to some embodiments;

FIG. 1G is a cross-sectional view of the portion of the catheter system, according to some embodiments;

FIG. 2A is an upper perspective view of the catheter system that includes hemolysis protection, illustrating another example blood collection adapter in a proximal position, according to some embodiments;

FIG. 2B is a cross-sectional view of a portion of the catheter system, illustrating the other blood collection adapter in the proximal position, according to some embodiments;

FIG. 2C is an exploded view of the portion of the catheter system, according to some embodiments;

FIG. 2D is a cross-sectional view of the portion of the catheter system, illustrating the other blood collection adapter in the distal position, according to some embodiments;

FIG. 2E is a cross-sectional view of the portion of the catheter system, illustrating an example male luer, according to some embodiments;

FIG. 2F is a cross-sectional view of an example distal end of the male luer, according to some embodiments;

FIG. 3A is a bar graph illustrating use of the blood collection adapter, according to some embodiments;

FIG. 3B is a bar graph illustrating use of the other blood collection adapter, according to some embodiments;

FIG. 3C is a bar graph illustrating use of the other blood collection adapter, according to some embodiments;

FIG. 4A is an upper perspective view of the catheter system that includes an example syringe, according to some embodiments;

FIG. 4B is a bar graph illustrating use of the syringe, according to some embodiments;

FIG. 5A is a side plan view of a blood collection adapter according to another aspect of the present disclosure;

FIG. 5B is a cross-sectional view of the blood adapter of FIG. 5A coupled to a connector in accordance with an aspect of the present disclosure;

FIG. 6A is a side plan view of the blood collection adapter of FIG. 5A coupled to a connector and a luer lock access device in accordance with an aspect of the present disclosure;

FIG. 6B is a cross-sectional view of the blood collection adapter, connector, and luer lock access device of FIG. 6A;

FIG. 7A is a side plan view of a luer lock access device in accordance with some embodiments;

FIG. 7B is a side plan view of another luer lock access device in accordance with some embodiments;

FIG. 7C is a side plan view of another luer lock access device in accordance with some embodiments;

FIG. 8A is a cross-sectional view of a blood collection adapter and connector in accordance with another aspect of the present disclosure;

FIG. 8B is a side plan view of a cannula of the blood collection adapter of FIG. 8A;

FIG. 8C is a partial cross-sectional view of the connector and cannula of the blood collection adapter of FIG. 8A;

FIG. 9A is a top plan view of a catheter system in accordance with an aspect of the present disclosure;

FIG. 9B is a side plan view of a blood collection adapter, connector, and luer lock access device in accordance with an aspect of the present disclosure;

FIG. 9C is a top plan view of the catheter system of FIG. 9A having the blood collection adapter, connector, luer lock access device, and blood collection container coupled thereto in accordance with another aspect of the present disclosure;

FIG. 9D is a top plan view of the catheter system of FIG. 9A having the blood collection adapter, connector, and syringe coupled thereto in accordance with another aspect of the present disclosure;

FIG. 9E is a top plan view of a catheter system in accordance with an aspect of the present disclosure;

FIG. 9F is a perspective view of the blood collection adapter of FIG. 9B in accordance with an aspect of the present disclosure;

FIG. 10A is a cross-sectional view of a blood collection adapter, connector, and luer lock access device in a first position in accordance with another aspect of the present disclosure;

FIG. 10B is a cross-sectional view of a blood collection adapter, connector, and luer lock access device in a second position in accordance with another aspect of the present disclosure;

FIG. 11A is a perspective view of a blood collection adapter in accordance with another aspect of the present disclosure;

FIG. 11B is a cross-sectional view of the blood collection adapter of FIG. 11A;

FIG. 12A is a perspective view of the blood collection adapter of FIG. 11A coupled to a luer lock access device in accordance with another aspect of the present disclosure;

FIG. 12B is a cross-sectional view of the blood collection adapter of FIG. 11A coupled to a luer lock access device and needleless connector in accordance with another aspect of the present disclosure;

FIG. 12C is a partial cross-sectional view of the blood collection adapter of FIG. 11A coupled to a needleless connector;

FIG. 13 is a partial cross-sectional view of a blood collection adapter and needleless connector in accordance with another aspect of the present disclosure;

FIG. 14A is a top plan view of a catheter system in accordance with another aspect of the present disclosure;

FIG. 14B is a side plan view of a blood collection adapter and luer lock access device in accordance with an aspect of the present disclosure;

FIG. 14C is a top plan view of the catheter system of FIG. 14A having the blood collection adapter, connector, and luer lock access device coupled thereto in accordance with another aspect of the present disclosure;

FIG. 14D is a top plan view of the catheter system of FIG. 14A having the blood collection adapter, connector, luer lock access device, and blood collection container coupled thereto in accordance with another aspect of the present disclosure;

FIG. 14E is a top plan view of the catheter system of FIG. 14A having the blood collection adapter, connector, and syringe coupled thereto in accordance with another aspect of the present disclosure;

FIG. 14F is a top plan view of a catheter system in accordance with an aspect of the present disclosure;

FIG. 14G is a perspective view of the blood collection adapter of FIG. 14B in accordance with an aspect of the present disclosure;

FIG. 15A is a cross-sectional view of a blood collection adapter, connector, and luer lock access device in a first position in accordance with another aspect of the present disclosure; and

FIG. 15B is a cross-sectional view of a blood collection adapter, connector, and luer lock access device in a second position in accordance with another aspect of the present disclosure.

### DESCRIPTION OF EMBODIMENTS

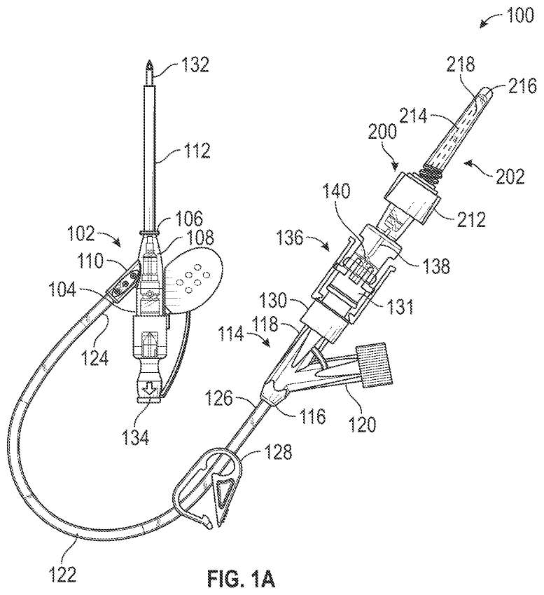

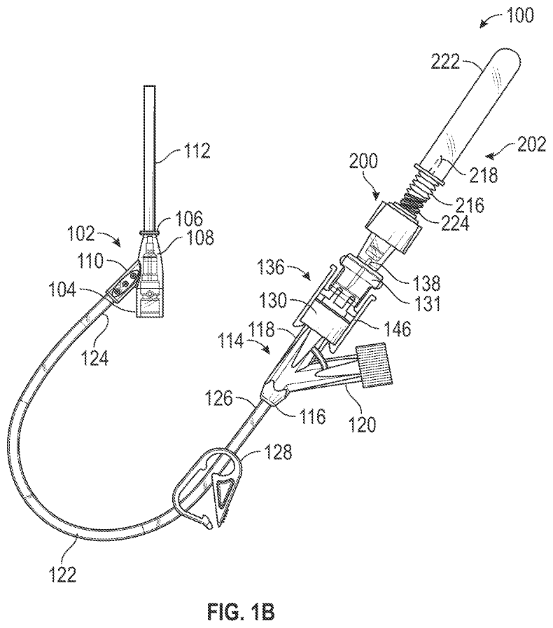

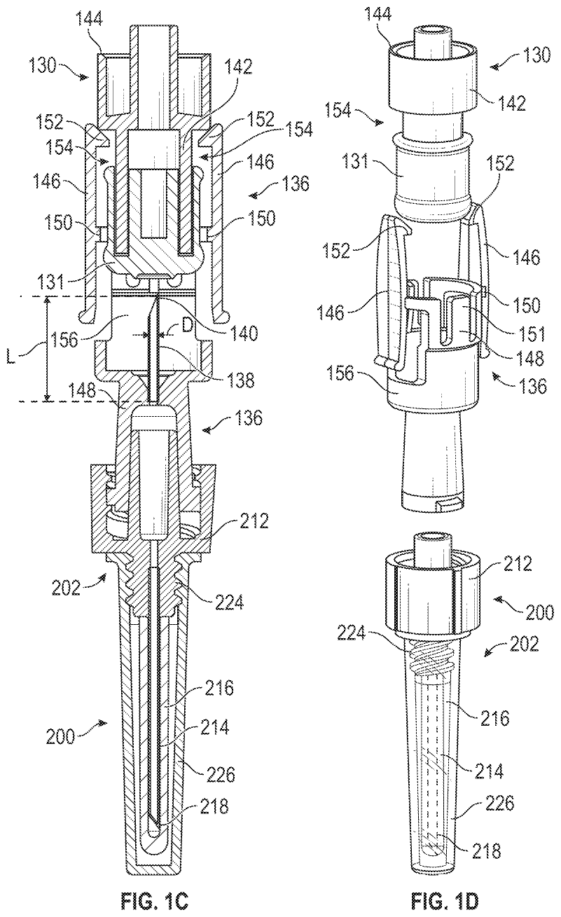

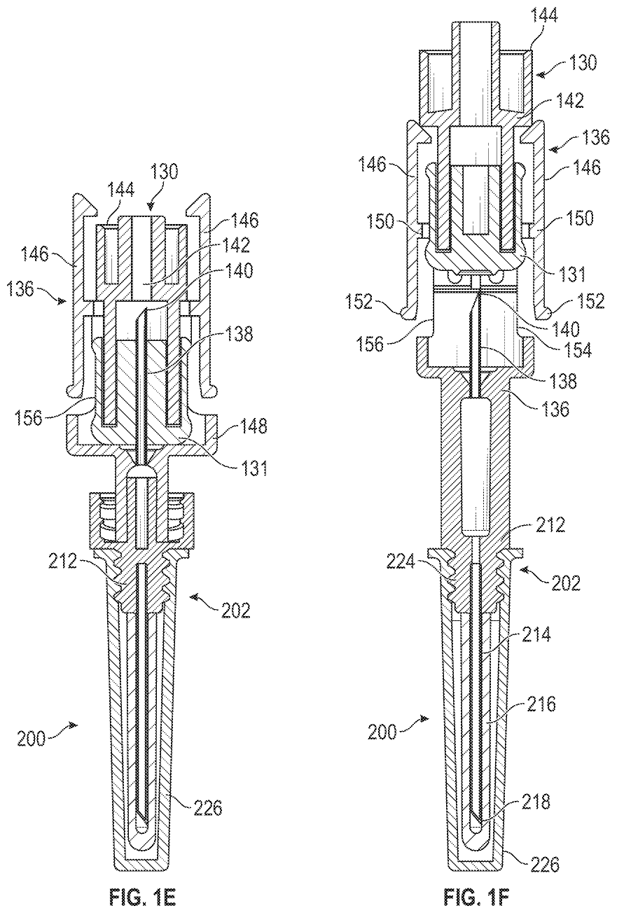

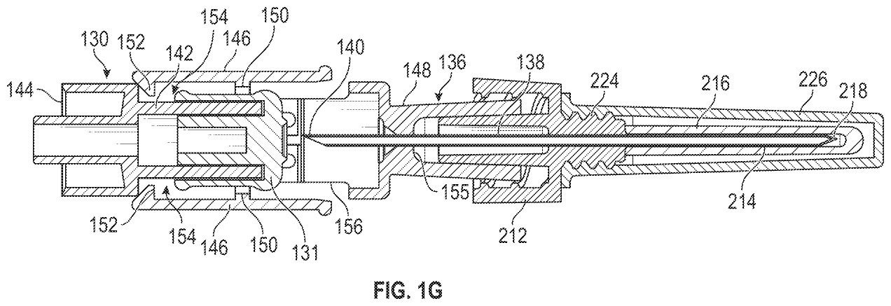

Referring now to FIGS. 1A-1B, in some embodiments, a catheter system 100 may include a catheter adapter 102, which may include a proximal end 104, a distal end 106, and a lumen 108 extending therebetween. In some embodiments, a side port 110 may be disposed between the distal end 106 of the catheter adapter 102 and the proximal end 104 of the catheter adapter 102. In these and other embodiments, a catheter 112 may extend distally from the distal end 106 of the catheter adapter 102. In some embodiments, the catheter 112 may include a peripheral intravenous catheter, a peripherally inserted central catheter, a midline catheter, or another suitable catheter.

In some embodiments, a catheter assembly of the catheter system 100 may include or correspond to any suitable catheter assembly, such as, for example, the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable catheter assembly.

In some embodiments, the catheter system 100 may include an adapter 114. In some embodiments, the adapter 114 may include a Y-adapter, a straight adapter, or another suitable adapter. In some embodiments, the adapter 114 may include one or more of a distal end 116, a first port 118, and a second port 120. In some embodiments, an extension tube 122 may be integrated with the adapter 114 and/or a portion of the catheter adapter 102, such as, for example, the side port 110. In some embodiments, in response to the catheter 112 being inserted into the vasculature of the patient, blood may flow through the catheter 112, at least a portion of the catheter adapter 102, the extension tube 122, and into the adapter 114.

In some embodiments, the catheter system 100 may include the extension tube 122, which may include a distal end 124 integrated with the side port 110 and/or a proximal end 126 integrated with the distal end 116 of the adapter 114. In some embodiments, a clamp, such as, for example, a pinch clamp 128, or another suitable clamp, may be disposed on the extension tube 122 and configured to selectively clamp the extension tube 122 to close a fluid path through the extension tube 122. In some embodiments, the pinch clamp 128 may be replaced with any suitable clamp.

In some embodiments, the first port 118 and/or the second port 120 of the adapter 114 may be coupled to a connector 130. In further detail, in some embodiments, the connector 130 may be monolithically formed as a single unit with the first port 118 and/or second port 120. In other embodiments, the first port 118 and/or the second port 120 of the adapter 114 may be coupled to the connector 130 via a luer connection. In these embodiments, the connector 130 may include a luer, such as, for example, a female or male luer. In some embodiments, the luer may be threaded or slip-fit with the adapter 114. In some embodiments, the connector 130 may be pre-attached to the adapter 114 during manufacture and assembly.

In some embodiments, the catheter 112 may include an over-the-needle catheter such that an introducer needle 132 extends from a needle hub 134 and through the catheter 112 to insert the catheter system 100 into the vasculature of the patient. In some embodiments, the needle hub 134 may be removably coupled to the proximal end 104 of the catheter adapter 102. In some embodiments, the catheter 112 and the introducer needle 132 may be assembled such that the sharp distal tip of the introducer needle 132 extends beyond the distal tip of the catheter 112, with the bevel of the introducer needle 132 facing up away from skin of the patient.

In some embodiments, the introducer needle 132 and the catheter 112 may be inserted at a shallow angle through the skin into the vasculature of the patient. In some embodiments, in response to the introducer needle 132 and the catheter 112 being inserted into the vasculature of the patient, blood may flow through the introducer needle 132 and into the needle hub 134, and the blood may be visible within the needle hub 134 to indicate to a user that the introducer needle 132 is within the vasculature of the patient.

In some embodiments, the connector 130 may be coupled to a blood collection adapter 136. In some embodiments, the blood collection adapter 136 may include a proximal end configured to receive a blood collection set 200. Some embodiments of the blood collection set 200 may include a needle assembly 202, which may be configured to engage the blood collection adapter 136. In some embodiments, the blood collection set 200 may be used for one or more of collecting blood from a patient, flushing, and infusion.

In some embodiments, a proximal end of the connector 130 may include a septum 131, which may be coupled to a body of the connector 130. In some embodiments, the blood collection adapter 136 may include a cannula 138 configured to penetrate or extend through the septum 131 to provide a fluid path through the connector 130. In these and other embodiments, a tip of the cannula 138 may be disposed within the blood collection adapter 136, which may decrease a risk of injury to a user. In some embodiments, the tip of the cannula 138 may be blunt, which may decrease a risk of injury to the connector 130, or sharp, which may facilitate penetration of the septum 131. In some embodiments, the cannula 138 may extend distally from the blood collection adapter 136, and a proximal end of the cannula 138 may be secured within the blood collection adapter 136 via a press fit, an adhesive, a mechanical device, or another suitable mechanism. In some embodiments, the cannula 138 may include a notch between the tip of the cannula 138 and a proximal end of the cannula. In some embodiments, the notch may provide access to a lumen of the cannula 138 and facilitate flushing after blood draw.

In some embodiments, the cannula 138 may be constructed of metal or another suitable material. In some embodiments, an outer diameter of the cannula 138 may be uniform along all or a portion of a length of the cannula 138. In some embodiments, a portion of the blood collection adapter 136 from which the cannula 138 extends may be constructed of plastic or another suitable material. In some embodiments, all or a portion of the blood collection adapter 136 may be constructed of plastic or another suitable material.

In some embodiments, the needle assembly 202 of the blood collection set 200 may include a body 212, a sharp needle 214 extending proximally from the body 212, and a sheath 216 covering a sharp proximal tip 218 of the sharp needle 214. In some embodiments, the sheath 216 may be elastomeric. In some embodiments, the sheath 216 may be plastic or another suitable material. In some embodiments, the sharp needle 214 may be secured within the body 212 via a press fit, an adhesive, a mechanical device, or another suitable mechanism. In some embodiments, the sharp needle 214 may extend proximally beyond the body 212.

In some embodiments, the sheath 216 may be coupled to the body 212 of the needle assembly 202. In some embodiments, the sharp proximal tip 218 of the sharp needle 214 may be enveloped within the sheath 216. In some embodiments, the sheath 216 may include an open distal end and a closed proximal end. In some embodiments, the body 212 may include a luer, which may be coupled to a corresponding luer of the proximal end of the blood collection adapter 136.

As illustrated in FIG. 1A, in some embodiments, the blood collection adapter 136 may be coupled to the connector 130 in a proximal position in which a distal tip 140 of the cannula 138 is in close proximity to or contacting the connector 130 without extending through or puncturing the connector 130 or the septum 131. In these and other embodiments, the distal tip 140 may be proximal to the septum 131. In this manner, some embodiments of the catheter system 100 may reduce a risk of leakage by maintaining a closed fluid path during a shelf-life of the catheter system 100. In some embodiments, the blood collection adapter 136 may be pre-attached to the connector 130 in the proximal position during manufacture and assembly. In other embodiments, the blood collection adapter 136 may be pre-attached to the connector 130 during manufacture and assembly with the cannula 138 extending through the septum 131 of the connector 130.

In some embodiments, the blood collection adapter 136 may be movable from the proximal position to a distal position, illustrated, for example, in FIG. 1B. In some embodiments, in response to the blood collection adapter 136 being in the distal position, a fluid path through the septum 131 may be opened. In some embodiments, the user may move the blood collection adapter 136 from the proximal position to the distal position to push the cannula 138 into and/or through the septum 131 prior to blood collection, for example.

In some embodiments, the blood collection set 200 may be configured to receive a blood collection device 222, such as a blood collection tube or BD VACUTAINER® blood collection tube, for example. In some embodiments, in response to the blood collection device 222 pushing the sheath 216 distally towards the body 212, the sharp needle 214 may pierce the sheath 216 and the sharp proximal tip 218 of the needle assembly 202 may be inserted into the blood collection device 222 when the blood collection adapter 136 is in the distal position.

In some embodiments, inserting the sharp proximal tip 218 into the blood collection device 222 may couple the blood collection device 222 to the blood collection set 200. In some embodiments, the blood collection device 222 may be coupled to the blood collection set 200 after the catheter 112 is positioned within the vasculature and/or the blood collection adapter 136 is moved to the distal position. In some embodiments, when the blood collection device 222 is penetrated by the sharp proximal tip 218, blood may flow into the blood collection device 222 due to a pressure differential between the vasculature and the blood collection device 222. In some embodiments, the needle assembly 202 may include threading 224, which may be coupled to corresponding threading of a cylindrical holder (not illustrated) for the blood collection device 222.

Referring now to FIG. 1C, the blood collection set 200 and the blood collection adapter 136 are illustrated coupled to the connector 130 and in the proximal position, according to some embodiments. In some embodiments, the proximal end of the connector 130 may include the septum 131, which may be coupled to the body 142 of the connector 130. In some embodiments, the body 142 may be rigid or semi-rigid. In some embodiments, the septum 131 may be constructed of an elastomeric and/or resilient material. In these and other embodiments, the septum 131 may include a slit or may not include the slit. In some embodiments, a distal end of the connector 130 may include a luer 144.

In some embodiments, the distal tip 140 of the cannula 138 may be surrounded by the blood collection adapter 136 to increase safety to the user. In some embodiments, the blood collection adapter 136 may include two or more lever arms 146, which may oppose each other and may each be connected to a body 148 of the blood collection adapter 136 at a flex point 150. In some embodiments, a hook member 152 disposed at a distal end of each of the lever arms 146 may be configured to be disposed within a groove 154 of the connector 130 when the lever arm 146 is in a relaxed position, as illustrated, for example, in FIG. 1C. In some embodiments, the groove 154 may be annular or the connector 130 may include separate grooves.

In some embodiments, a proximal end of each of the lever arms 146 proximal to the flex point 150 may be pressed inwardly to bias a distal end of the lever arm 146 outwardly and release the hook member 152 of the lever arm 146 from the groove 154. In some embodiments, the distal end of each of the lever arms 146 may automatically return from the biased positioned to the relaxed position in response to the proximal end of the lever arm 146 being released or not pressed inwardly.

In some embodiments, the blood collection adapter 136 may include one or more fingers 151 which may be configured to bend or bias outwardly to fit over the connector 130. In some embodiments, the fingers 151 may extend in a distal direction from a cylindrical portion 156 of the body 148. In some embodiments, the distal tip 140 of the cannula 138 may be disposed within the cylindrical portion 156 to prevent an accidental needle stick injury.

In some embodiments, the blood collection adapter 136 and the body 212 may be removably coupled together and separable. In these and other embodiments, the blood collection set 200 may be used for infusion after blood collection via the needle assembly 202 and removal of the needle assembly 202 from the blood collection adapter 136. Referring now to FIG. 1F, the blood collection adapter 136 and the body 212 of the needle assembly 202 may be integrated or monolithically formed as a single unit.

Referring back to FIG. 1C, in some embodiments, the blood collection set 200 may include a cover 226, which may be removed to couple a particular blood collection container.

An entire length of the cannula 138 may be represented by L, and an inner diameter of the cannula 138 may be represented by D. In some embodiments, the inner diameter of the cannula 138 may be constant and/or greater than a minimum inner diameter of the catheter 112 (see FIGS. 1A-1B, for example). In some embodiments, D⁴/L is equal to or less than a predetermined value to reduce maximum shear stress. In some embodiments, the predetermined value of D⁴/L may be as follows:

| | Upper limit of D\hat{ }4/L to match | Upper limit of D\hat{ }4/L to match |
|---|---|---|
| Gauge | 21G UT (in\hat{ }3) | 25G UT (in\hat{ }3) |
| 18G | 3.10E−07 | 7.38E−07 |
| 20G | 4.93E−07 | 6.83E−06 |
| 22G | 3.60E−07 | 1.98E−06 |
| 24G | 7.59E−08 | 1.33E−07 |

In the table above, “21G UT” and “25G UT” refer, respectively, to a 21-gauge BD VACUTAINER® ULTRATOUCH™ push button blood collection set, and a 25-gauge BD VACUTAINER® ULTRATOUCH™ push button blood collection set. “Gauge” corresponds to a gauge of a catheter of a catheter assembly, such as the catheter 112 of FIGS. 1A-1B, for example.

Referring now to FIG. 1D, an exploded view of the connector 130, the blood collection adapter 136, and the blood collection set 200 is illustrated, according to some embodiments.

Referring now to FIG. 1E, the blood collection set 200 and the blood collection adapter 136 in the distal position, according to some embodiments. In some embodiments, in response to the blood collection adapter 136 moving from the proximal position to the distal position, the cannula 138 may penetrate or extend through the septum 131 to provide the fluid path through the connector 130.

Referring now to FIG. 1F, in some embodiments, the blood collection adapter 136 and the body 212 of the blood collection set 200 may be monolithically formed as a single unit, which may reduce a risk of the distal tip 140 of cannula 138 accidentally sticking a user. Referring now to FIG. 1G, in some embodiments, the cannula 138 may extend proximally to the sharp needle 214. In these embodiments, the cannula 138 and the sharp needle 214 may be monolithically formed as a single unit or joined together. In these embodiments, D may correspond to an inner diameter of the cannula 138 and may be constant. In some embodiments, L may correspond to the length of the cannula 138 or a length of the cannula 138 and the sharp needle 214 combined. In some embodiments, the length of the cannula 138 may extend from the distal tip 140 of the cannula 138 to or near a distal end 155 of an inner cavity.

In some embodiments, the predetermined value of D⁴/L may be as follows:

| | Upper limit of D\hat{ }4/L to match | Upper limit of D\hat{ }4/L to match |
|---|---|---|
| Gauge | 21G UT (in\hat{ }3) | 25G UT (in\hat{ }3) |
| 18G | 3.10E−07 | 7.38E−07 |
| 20G | 4.93E−07 | 6.83E−06 |
| 22G | 3.60E−07 | 1.98E−06 |
| 24G | 7.59E−08 | 1.33E−07 |

“21G UT” and “25G UT” refer to the BD VACUTAINER® ULTRATOUCH™ push button blood collection set used with a 21G catheter and a 25G catheter, respectively. The BD VACUTAINER® ULTRATOUCH™ push button blood collection set is available from Becton, Dickinson & Company of Franklin Lakes, New Jersey. “Gauge” corresponds to a gauge of a catheter of a catheter assembly, such as the catheter 112 of FIGS. 1A-1B, for example.

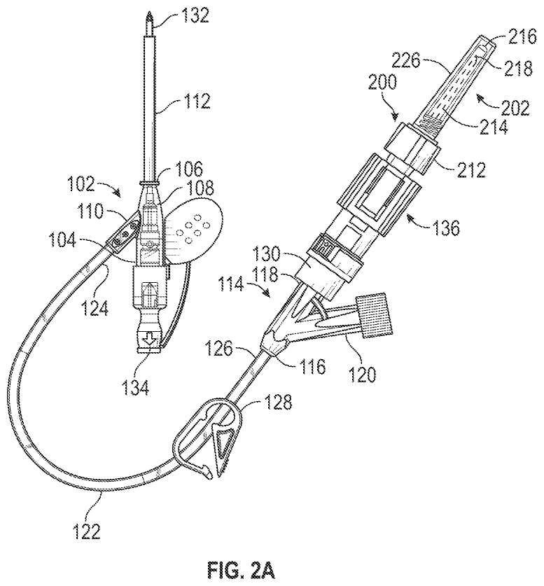

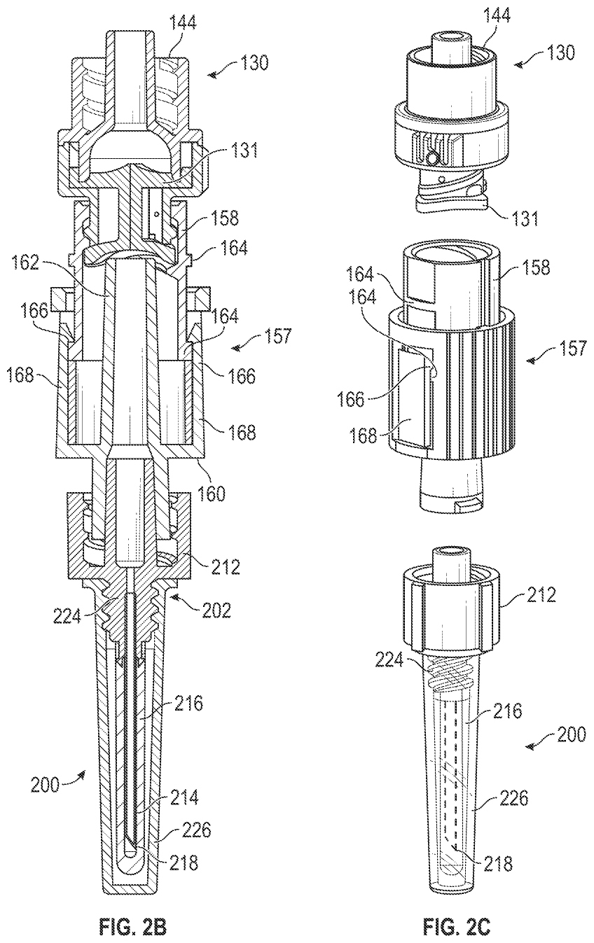

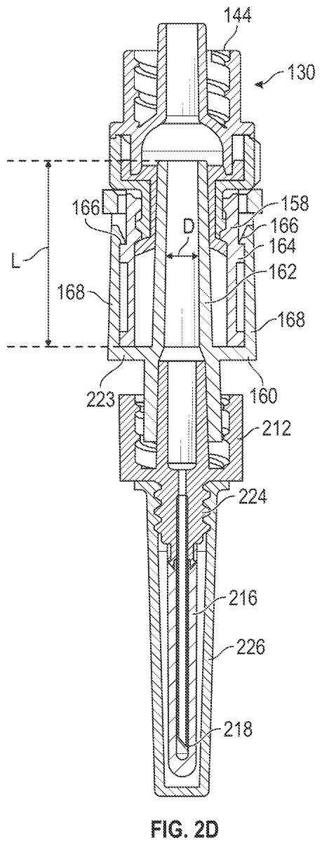

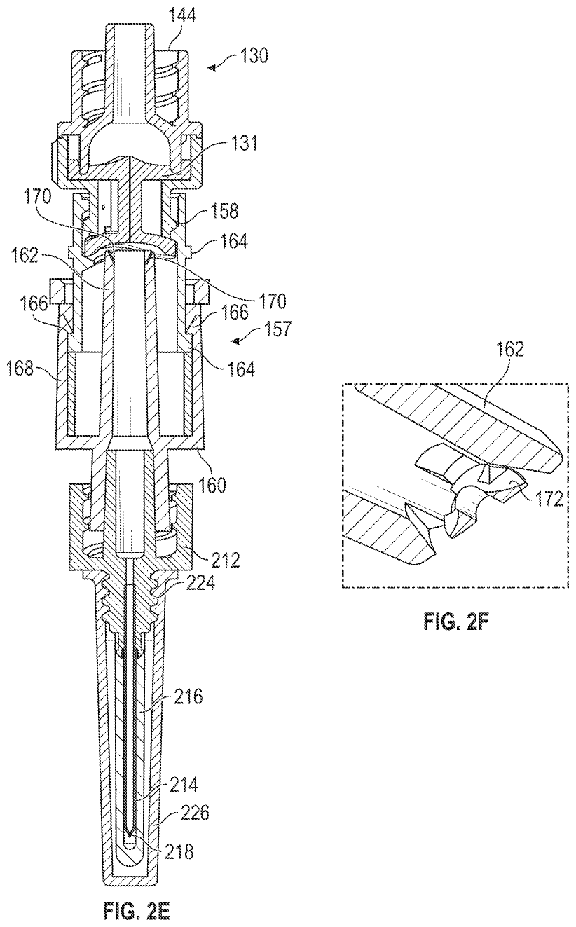

Referring now to FIGS. 2A-2D, in some embodiments, a blood collection adapter 157 may include a distal piece 158 and a proximal piece 160 movable distally towards the distal piece 158. In some embodiments, the blood collection adapter 157 may be similar or identical to the blood collection adapter 136 in terms of one or more features and/or operation.

In some embodiments, the proximal piece 160 of the blood collection adapter 157 may include a male luer, which may include an insert portion 162 and/or a surrounding collar. As illustrated, for example, in FIG. 2D, in some embodiments, the proximal piece 160 may be moved distally towards the distal piece 158. In some embodiments, the insert portion 162 may open or activate the septum 131 and create the fluid path through the connector 130 in response to the proximal piece 160 moving distally towards the distal piece 158. In some embodiments, in response to the proximal piece 160 moving distally towards the distal piece 15, the insert portion 162 may extend through the septum 131.

In some embodiments, the connector 130 may include a needleless connector. In some embodiments, the connector 130 may include, for example, a MAXPLUS™ needleless connector, a MAXZERO™ needleless connector, a BD Q-SYTE™ luer activated split septum, (available from Becton, Dickinson and Company of Franklin Lakes, New Jersey) a SMARTSITE™ needle-free connector (available from Becton, Dickinson and Company of Franklin Lakes, New Jersey) or another suitable connector. In some embodiments, the insert portion 162 may be monolithically formed with a collar portion the male luer, which may include threads or may be configured for a slip-fit.

In some embodiments, an outer surface of the distal piece 158 may include one or more protrusions 164, which may facilitate securement of the proximal piece 160 in a proximal position, illustrated, for example, in FIG. 2B, and/or a distal position, illustrated, for example, in FIG. 2D. In some embodiments, an inner surface of the blood collection adapter 157 may include one or more other protrusions 166 configured to slide past the protrusions 164 and engage the proximal piece 160 in a snap-fit with the distal piece 158. In some embodiments, the other protrusions 166 may be disposed on an inner surface of one or more arms 168 of the proximal piece 160 of the blood collection adapter 157, which may be detached from a remaining portion of the proximal piece 160 along opposing edges and an end of the arm 221 to provide flexing.

In these embodiments, D may correspond to an inner diameter of the insert portion 162, which may be constant. In some embodiments, L may correspond to a length of insert portion 162, which may extend from a base 223, from which the arms 168 may extend.

In some embodiments, the predetermined value of D⁴/L may be as follows:

| | Upper limit of D\hat{ }4/L to match | Upper limit of D\hat{ }4/L to match |
|---|---|---|
| Gauge | 21G UT (in\hat{ }3) | 25G UT (in\hat{ }3) |
| 18G | 3.10E−07 | 7.38E−07 |
| 20G | 4.93E−07 | 6.83E−06 |
| 22G | 3.60E−07 | 1.98E−06 |
| 24G | 7.59E−08 | 1.33E−07 |

“21G UT” and “25G UT” refer to the BD VACUTAINER® ULTRATOUCH™ push button blood collection set used with a 21G catheter and a 25G catheter, respectively. The BD VACUTAINER®) ULTRATOUCH™ push button blood collection set is available from Becton, Dickinson & Company of Franklin Lakes, New Jersey. “Gauge” corresponds to a gauge of a catheter of a catheter assembly, such as the catheter 112 of FIG. 2A, for example.

Referring now to FIG. 2E, in some embodiments, a distal end of the insert portion 162 may include one or more chamfers 170, which may facilitate flushing after blood draw. Referring now to FIG. 2F, in some the distal end of the insert portion 162 may include one or more flow diverters. For example, the distal end of the insert portion 162 may include a flow diverter 172 extending into a lumen of the insert portion 162. In some embodiments, the flow diverter may be configured to direct fluid flow to a peripheral circumferential dead space located distal to the distal end of the insert portion 162.

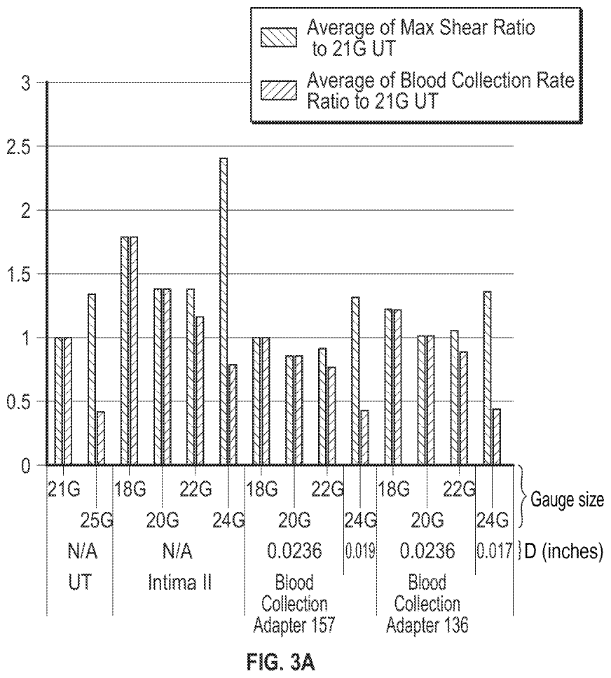

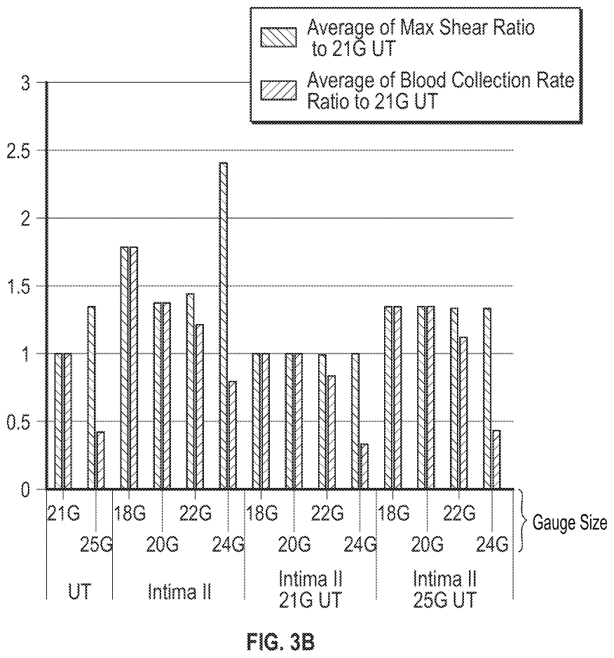

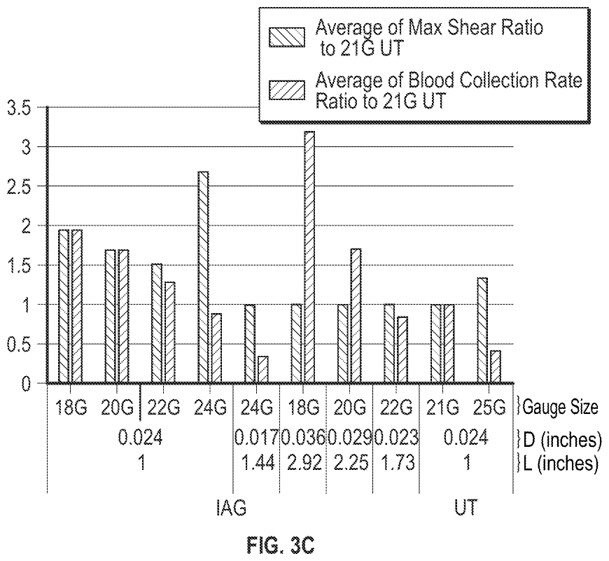

Referring now to FIG. 3A, in some embodiments, the blood collection adapter 136 and the blood collection adapter 157 may provide hemolysis protection, as mentioned with respect to FIGS. 1C and 2E. In further detail, the blood collection adapter 136 and the blood collection adapter 157 may limit a maximum blood collection rate, which in turn may limit a maximum shear stress during blood collection and reduce hemolysis. In some embodiments, the blood collection adapter 136 and the blood collection adapter 157 may be configured to limit a maximum shear stress that blood cells are exposed to during a blood draw to a predetermined or target value. In some embodiments, the fluid path extending through the blood collection adapter 136 or the blood collection adapter 157 may facilitate reduction of maximum shear stress to an acceptable level, reducing a likelihood of mechanical hemolysis.

“UT” on the x-axis of FIG. 3A refers to a prior art blood collection set, namely the BD VACUTAINER® ULTRATOUCH™ push button blood collection set. “Intima II” on the x-axis of FIG. 3A refers to the BD INTIMA II™ IV Catheter, a prior art catheter assembly used for blood draw without the blood collection adapter 136. “Blood Collection Adapter 136” refers to the blood collection adapter 136 referred to with respect to FIG. 1A-1F or a similar blood collection adapter used with a pro-re-nata (“PRN”) style connector. “Blood Collection Adapter 157” refers to the blood collection adapter 157 referred to with respect to FIG. 2A-2F or a similar adapter used with a needleless connector. “21G UT” refers to a 21G BD VACUTAINE® ULTRATOUCH™ push button blood collection set. FIG. 3A illustrates that use of the blood collection adapter 136 or blood collection adapter 157 may reduce the maximum shear stress to a same amount as a 25G BD VACUTAINER® ULTRATOUCH™ push button blood collection set.

Referring now to FIG. 3B, “UT” on the x-axis of FIG. 3B refers to a prior art blood collection set, namely the BD VACUTAINER® ULTRATOUCH™ push button blood collection set. “Intima II” on the x-axis of FIG. 3B refers to the BD INTIMA II™ IV Catheter, a prior art catheter assembly used for blood draw without the blood collection adapter 136 or the blood collection adapter 157. “Intima II 21G UT” on the x-axis of FIG. 3B refers to the BD INTIMA II™ IV Catheter with a fluid path matching “21G UT”. “Intima II 25G UT” on the x-axis of FIG. 3B refers to the BD INTIMA II™ IV Catheter with a fluid path matching “25G UT”. The blood collection adapter 136 is used with “Intima II 25G UT”, and the blood collection adapter 157 is used with “Intima II 21G UT”. 18G, 20G, 22G, or 24G correspond to a gauge of a catheter of a catheter assembly coupled to the blood collection adapter 136 or the blood collection adapter 157. FIG. 3B illustrates that use of the blood collection adapter 157 and the blood collection device 136 may reduce the maximum shear stress to a same amount as a 25G BD VACUTAINER® ULTRATOUCH™ push button blood collection set for every gauge of catheter.

Referring now to FIG. 3C, in some embodiments, dimensions of the fluid path of the blood collection adapter 136 (see, for example, FIGS. 1A-1G) may be selected to match the maximum shear stress to that of 21G BD VACUTAINER® ULTRATOUCH™ push button blood collection set. In FIG. 3C, “IAG” on the x-axis of FIG. 3C refers to BD INSYTE™ AUTOGUARD™ BC Shielded IV Catheter with Blood Control Technology, available from Becton, Dickinson & Company of Franklin Lakes, New Jersey. A similar result is expected for Intima II (BD INTIMA II™ IV Catheter available from Becton, Dickinson & Company) since the extension tube contributes little to fluidic resistance. D may correspond to an inner diameter of the cannula 138, which may equal an inner diameter of the sharp needle 214. L may correspond to a sum of the length of the cannula 138 and the sharp needle 214. In this case, the blood collection adapter 136 may facilitate improved blood collection rate while still matching the max shear stress of 21G or 25G “UT” (BD VACUTAINER® ULTRATOUCH™ push button blood collection set). In some embodiments, a blood collection rate for a 20G catheter and an 18G catheter can be increased significantly while matching the max shear stress of the 21G BD VACUTAINER® ULTRATOUCH™ push button blood collection set, as illustrated in FIG. 3C.

While the FIG. 3C discloses embodiments in which the inner diameter of the cannula 138 and the inner diameter of sharp needle 214 are equal, in accordance with other embodiments of the present disclosure, the gauge (and, thus, the inner diameter) of cannula 138 and the gauge (and inner diameter) of the sharp needle 214 may be different, and may be selected specifically to optimize blood collection rate and hemolysis reduction for blood draws via a particular catheter assembly. For example, referring to the table below, various gauge combinations for cannula 138 and sharp needle 214 may be chosen based on the gauge of the catheter assembly, with the gauge combinations of the cannula 138 and sharp needle 214 resulting in a combined hydraulic resistance:

| Catheter Gauge | Cannula 138 and Sharp Needle 214 Combined |
|---|---|
| (w/length) | Hydraulic Resistance (Pa*s/m³) |
| 18G (1.16 in.) | 6.79e+10 |
| 20G (1.16 in) | 6.52e+10 |
| 22G (1.0 in) | 6.38e+10 |
| 24G (0.75 in) | 1.51e+11 |

As discussed above, 18G, 20G, 22G, or 24G correspond to the gauge of the catheter assembly coupled to the blood collection adapter 136. The respective type and dimensions of the cannula 138 and sharp needle 214 of the blood collection adapter 136 may be selected based on the gauge of the catheter assembly, with the cannula and/or sharp needle being chosen to optimize volumetric flow rate, while also accounting for acceptable shear-induced hemolysis of the blood sample. Such a determination may be made by any appropriate method, such as computational fluid dynamics (CFD) simulations, bench testing, etc.

In the example shown above, for both the 18G and 20G catheter assembly, it was determined that optimized flow and hemolysis conditions were possible with the cannula 138 and sharp needle 214 being of the same gauge. However, for the 22G and/or 24G catheter assemblies, optimized flow and hemolysis conditions were possible with modification of the combined hydraulic resistances of cannula 138 and sharp needle 214 having different gauges. It is to be understood that the above examples represent only one embodiment, and that different gauges and/or cannula dimensions (and, thus, different combined hydraulic resistances) may be selected for other catheter assemblies.

Furthermore, while not shown in the table above, the length of the cannula 138 may also be selected based on a determined hemolysis potential of the blood sample and the blood collection rate. Additionally, and/or alternatively, the cannula 138 and/or the sharp needle 214 may be selected to have different wall thicknesses to further reduce hemolysis potential and/or flow rate. For example, one or both of the cannula 138 and the sharp needle 214 may have a regular wall (or “RW”) or thin wall (or “TW”) thickness, with the thickness of the cannula determining the inside diameter of the cannula and, thus, altering the hemolysis potential and/or flow rate.

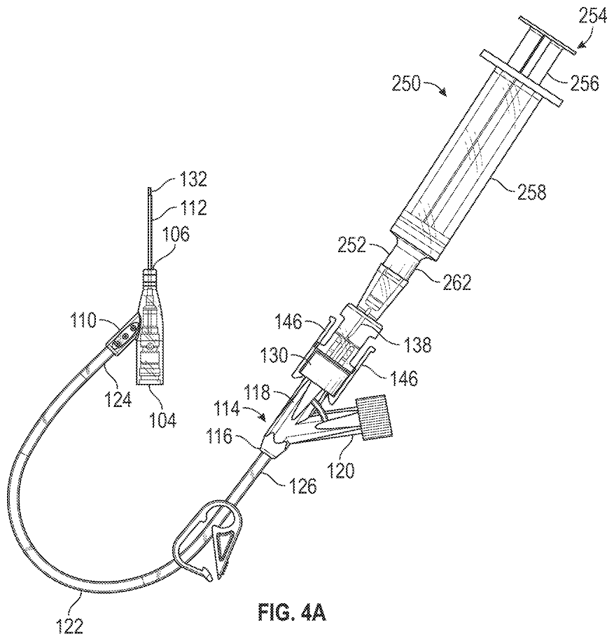

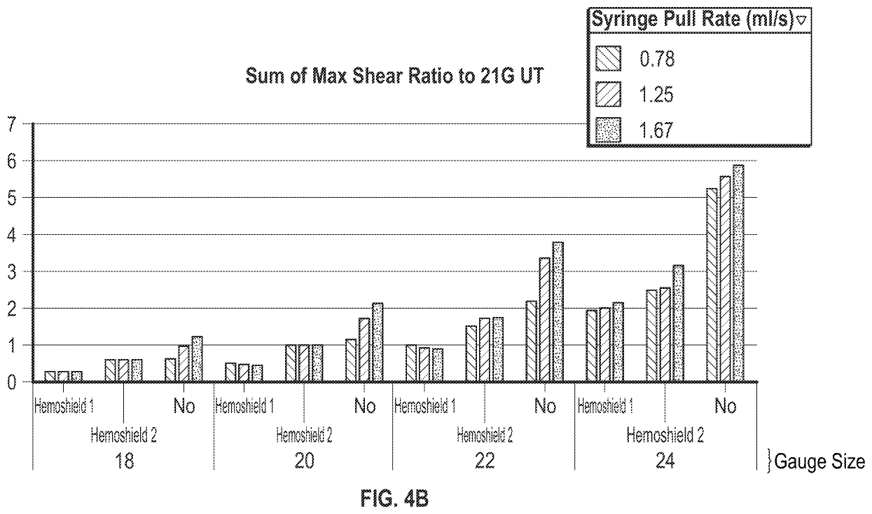

Referring now to FIG. 4A, in some embodiments, the catheter system 100 may include a syringe 250, which may include a distal end 252 and a proximal end 254. In some embodiments, the syringe 250 may include a sliding plunger 256 that fits tightly in a barrel or tube 258. In some embodiments, the sliding plunger 256 may be pulled proximally with respect to the tube 258, allowing the syringe 250 to draw in a liquid, such as blood, through an orifice at the distal end 252. In some embodiments, the sliding plunger 256 may be pushed distally with respect to the tube 258, allowing the syringe 250 to then expel the liquid through the orifice. In some embodiments, the syringe 250 may be coupled to a proximal end of a particular blood collection adapter, such as the blood collection adapter 136 or the blood collection adapter 157, which may include the luer 262, which may include a female luer. The blood collection adapter 136 is illustrated in the distal position, according to some embodiments.

Referring now to FIG. 4B, for a 20G catheter, the risk of hemolysis is moderate at low syringe pull rates but increases at higher pull rates. With a blood collection adapter having D⁴/L of 2.7 e−7, the maximum shear stress with a catheter draw is reduced to the maximum shear stress of 21G UT (BD VACUTAINER® ULTRATOUCH™ push button blood collection set with a 21G catheter) for all tested syringe draw rates. For a 22G catheter, the risk of hemolysis is significant at higher draw rates. A blood collection adapter with D⁴/L of 7 e−8 again reduces the maximum shear stress to that of the gold standard (BD VACUTAINER®) ULTRATOUCH™ push button blood collection set with a 21G catheter) for all tested syringe pull rates. For a 24G catheter, the above two blood collection adapters are able to reduce the max shear stress 2.5 to 3 times depending on the syringe draw rate. The resultant effects on maximum shear stress are illustrated in FIG. 4B, according to some embodiments.

As noted above, after blood draw, it is generally desirable to flush the adapter 114 and/or connector 130 with, e.g., a saline solution so as to remove any residual blood remaining therein. However, conventional blood draw techniques via indwelling catheters often utilize an elongated needle cannula fluidly coupled to an extension set, with the elongated needle cannula being configured to pierce the septum 131 in order to fluidly couple the extension set to the connector 130. The distal tip of such an elongated needle cannula generally extends far into the interior of connector 130, leaving substantial amounts of “dead space” within the connector 130 at locations proximal to the distal tip of the needle cannula. As such, the flushing of residual blood remaining within the connector 130 using such a configuration is often not sufficient, as the dead space within the connector 130 is not fully accessed by the flushing fluid ejected from the distal tip of the needle cannula. Furthermore, use of such an elongated needle cannula may increase the likelihood of needle stick injuries to the care provider(s) and/or patient.

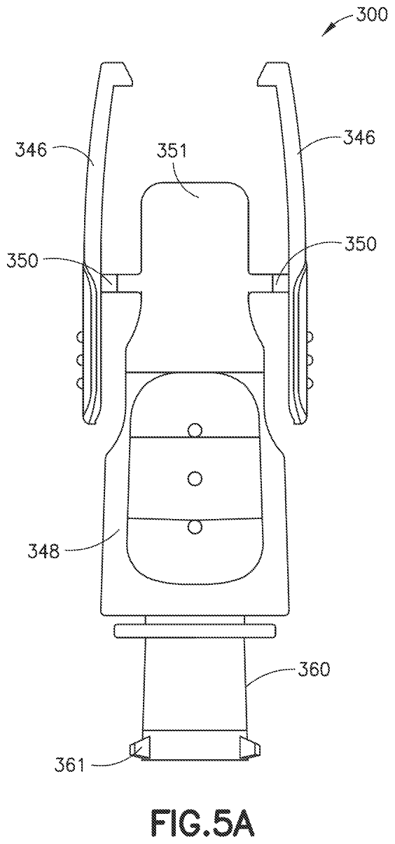

Thus, in accordance with another aspect of the present disclosure, and referring to FIGS. 5A and 5B, a blood collection adapter 300 configured for improved flushing and a reduced likelihood of needle stick injuries is disclosed.

As shown in FIG. 5B, a connector 330 may be coupled to a blood collection adapter 300. It is to be understood that connector 330 is substantially similar to connector 130 described above. Furthermore, as will be described below, the blood collection adapter 300 may include a proximal end configured to receive a blood collection set. In some embodiments, the blood collection set may be used for one or more of collecting blood from a patient, flushing, and infusion.

In some embodiments, a proximal end of the connector 330 may include a septum 331, which may be coupled to a body of the connector 330. The blood collection adapter 300 may include a cannula 338 configured to penetrate or extend through the septum 331 to provide a fluid path through the connector 330. In these and other embodiments, a tip 340 of the cannula 338 may be disposed within the blood collection adapter 300, which may decrease a risk of needle-stick injury to a user. In some embodiments, the distal tip 340 of the cannula 338 may be blunt, which may decrease a risk of injury, or sharp, which may facilitate penetration of the septum 331. In some embodiments, the cannula 338 may extend distally from the blood collection adapter 300, and a proximal end of the cannula 338 may be secured within the blood collection adapter 300 via a press fit, an adhesive, a mechanical device, or another suitable mechanism. For example, as is shown in FIGS. 5A and 5B, the cannula 338 may be secured to an adapter hub 360, with the adapter hub 360 having an inner body portion 355 surrounding a portion of the cannula 338 and a proximal coupling portion 361 configured to secure the adapter hub 360 to, e.g., a luer lock access device. While shown in FIGS. 5A and 5B as a separate component, it is to be understood that, in some embodiments, the adapter hub 360 may be monolithically formed with the blood collection adapter 300.

In some embodiments, a portion of the blood collection adapter 300 from which the cannula 338 extends may be constructed of plastic or another suitable material. In some embodiments, all or a portion of the blood collection adapter 300 may be constructed of plastic or another suitable material.

As is shown in FIG. 5B, the septum 331 of the connector 330 is coupled to a body 342 of the connector 330. In some embodiments, the body 342 may be rigid or semi-rigid. In some embodiments, the septum 331 may be constructed of an elastomeric and/or resilient material. In these and other embodiments, the septum 331 may include a central slit or may not include the slit. Furthermore, similar to connector 130 disclosed above, a distal end of the connector 130 may include a luer connector.

In some embodiments, the distal tip 340 of the cannula 338 may be surrounded by the blood collection adapter 300 to increase safety to the user. Additionally, the blood collection adapter 300 may include two or more lever arms 346, which may oppose each other and may each be connected to a body 348 of the blood collection adapter 300 via a flex point 350. In some embodiments, a hook member similar to hook member 152 described above with respect to adapter 136 may be disposed at a distal end of each lever arms 346 so as to engage with a groove of the connector 330 when the lever arm 346 is in a relaxed position, thereby releasably coupling the blood collection adapter 300 to the connector 330.

In some embodiments, a proximal end of each of the lever arms 346 proximal to the flex point 350 may be pressed inwardly to bias a distal end of the lever arm 346 outwardly and release the hook member of the lever arm 346 from the connector 330. In some embodiments, the distal end of each of the lever arms 346 may automatically return from the biased positioned to the relaxed position in response to the proximal end of the lever arm 346 being released or not pressed inwardly.

As is shown in FIG. 5A, the blood collection adapter 300 may include one or more side members 351, which may be configured to fit over and/or at least partially around the connector 330. In some embodiments, the one or more side members 351 may be flexible and/or resilient so as to accommodate the septum 331. In some embodiments, the one or more side members 351 may extend in a distal direction from the body 348 of the blood collection adapter 300, thereby at least partially shielding the distal tip 340 of the cannula 338 to aid in the prevention of accidental needle stick injuries.

Referring again to FIG. 5B, the cannula 338 is shown in a fully distal position relative to the connector 330, wherein the distal tip 340 extends through or punctures the septum 331 and is in communication with an interior passage 352 of the connector 330. However, the distal tip 340 of cannula 338 is substantially limited in the distance within the interior passage 352 through which it can travel due to the interface between the adapter hub 360 and the body 348, thereby maintaining the distal tip 340 (and the beveled portion of cannula 338 immediately below distal tip 340) at a position substantially within a proximal portion of the interior passage 352. In this way, not only can cannula 338 be used to effectively draw blood through connector 330, but the positioning of distal tip 340 within the interior passage 352 of connector 330 allows for residual blood to be effectively flushed from the connector 330 after blood draw, as the dead space proximal to the distal tip 340 within the interior passage 352 is minimized.

While not shown or described in detail, similar to cannula 138 described above, it is to be understood that cannula 338 may be configured to have an internal diameter(s) and length optimized so as to reduce shear stress and, thus, reduce hemolysis during blood draw.

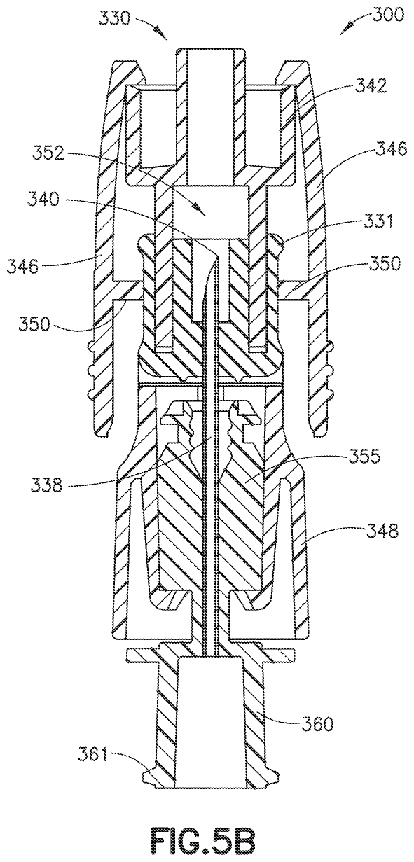

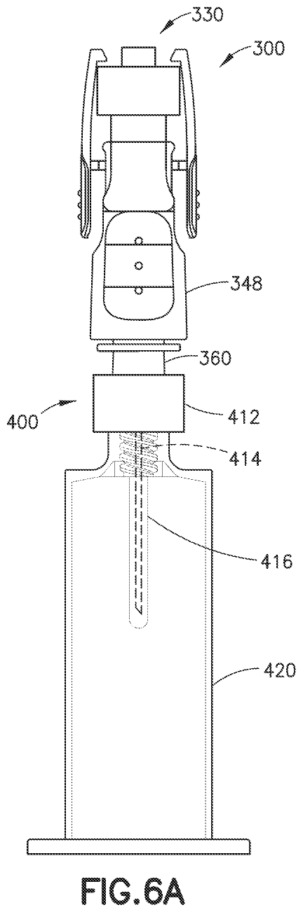

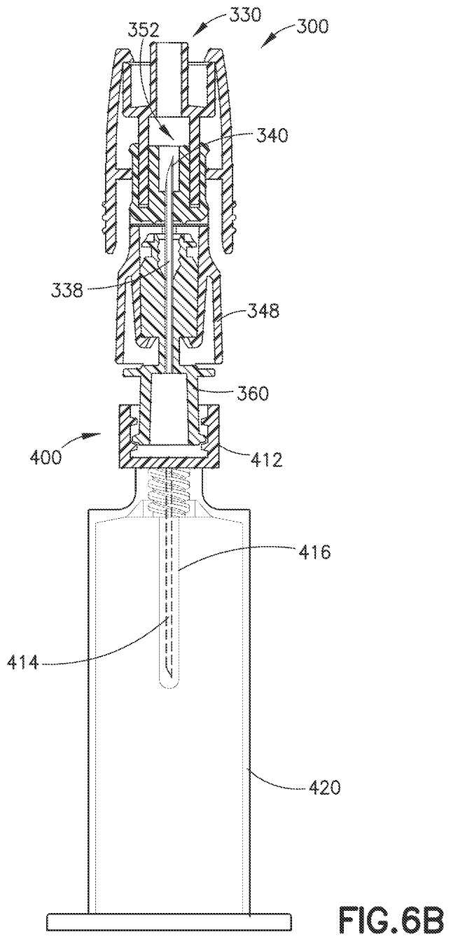

Referring now to FIGS. 6A and 6B, in some embodiments, the blood collection adapter 300 may be configured to be coupled to a luer lock access device 400 so as to receive a blood collection device such as, e.g., a BD VACUTAINER® blood collection tube. The luer lock access device 400 includes a luer lock hub 412 having internal threads capable of engaging the proximal coupling portion 361 of the adapter hub 360 to releasably secure the blood collection adapter 300 to the luer lock access device.

The luer lock access device 400 further includes a needle 414 in fluid communication with the luer lock hub 412 so as to allow fluids to pass from the blood collection adapter 300 to the blood collection device. The needle 414 may be substantially surrounded by a sheath 416 to protect from needle stick injuries. The sheath 416 may be formed of rubber or any other appropriate material. Furthermore, the luer lock access device 400 may include a holder 420, which is sized and configured to accommodate a blood collection device such as, e.g., a BD VACUTAINER® blood collection tube. In response to the blood collection device pushing the sheath 416 distally towards the body luer lock hub 412, the needle 414 may pierce the sheath 416 and the sharp proximal tip of the needle 414 may be inserted into the blood collection device so as to receive a blood sample via the blood collection adapter 300.

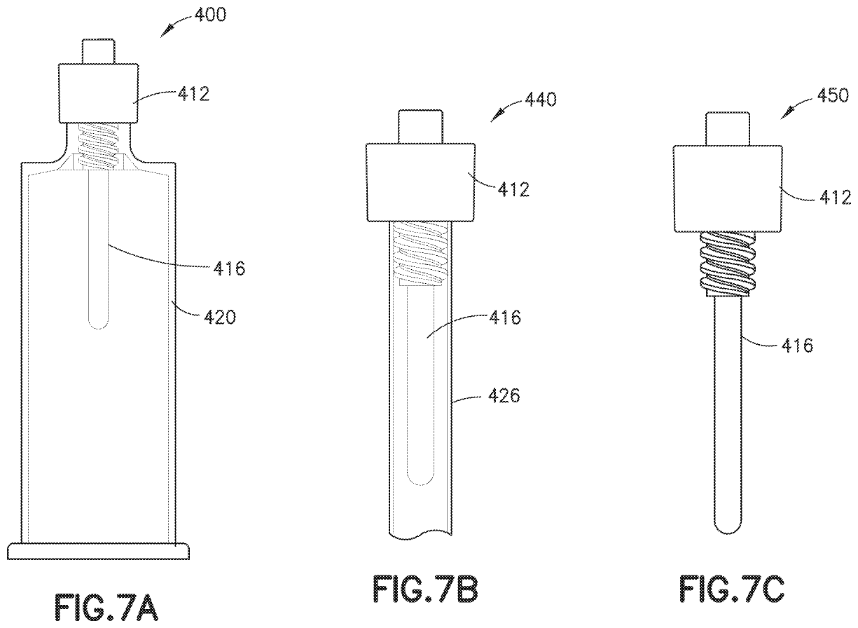

Referring to FIGS. 7A-7C, it is to be understood that the luer lock access device in accordance with embodiments of the present disclosure may have various configurations. For example, as shown in FIG. 7A (as well as FIGS. 6A and 6B), the luer lock access device 400 includes the luer lock hub 412, needle 414, sheath 416, and holder 420. However, as shown in FIG. 7B, a luer lock access device 440 in accordance with another embodiment may be utilized, wherein the holder is omitted, but an additional protective cover 426 is utilized around the needle 414 and sheath 416. The protective cover 426 may be selectively removable by the user in order to couple a blood collection device to the needle 414. Further still, referring to FIG. 7C, a luer lock access device 450 may be provided, with the luer lock access device 450 including only the luer lock hub 412, needle 414, sheath 416.

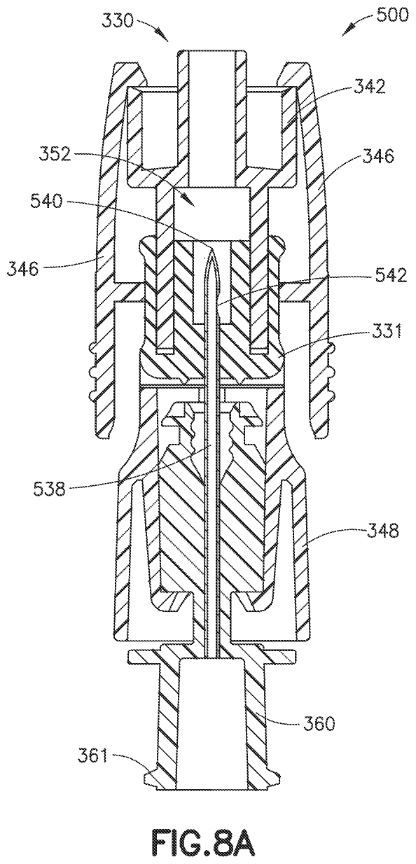

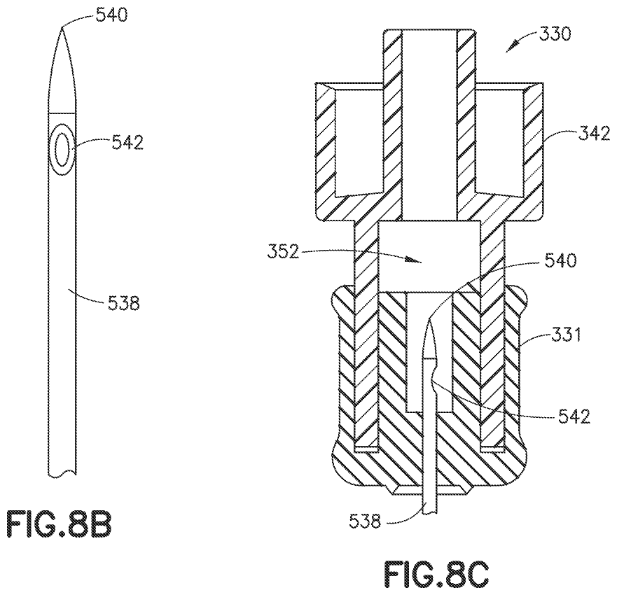

Next, referring to FIGS. 8A-8C, a blood collection adapter 500 in accordance with another aspect of the present disclosure is illustrated. As shown in FIG. 8A, blood collection adapter 500 includes many of the same features as blood collection adapter 300 shown and described above with respect to FIGS. 5A and 5B. As such, the purpose and function of like components between blood collection adapter 300 and blood collection adapter 500 will not be reiterated herein.

However, unlike blood collection adapter 300, which utilizes the cannula 338 having a beveled opening proximate the tip 340, blood collection adapter 500 includes a cannula 538 having a side hole 542 positioned proximally to a tip 540. As shown in FIGS. 8A and 8C, the side hole 542 places the cannula 538 in fluid communication with a proximal portion of the interior passage 352 of connector 330. While the side hole 542 allows blood to be drawn through the cannula 538, its positioning relative to the interior passage 352 is particularly useful during flushing, as the fluid expelled from the side hole 542 may generate a turbulent flow within the connector 330 so as to improve the flush in the “dead space” of the connector 330, thereby cleaning any residual blood remaining in the connector 330 after a blood draw.

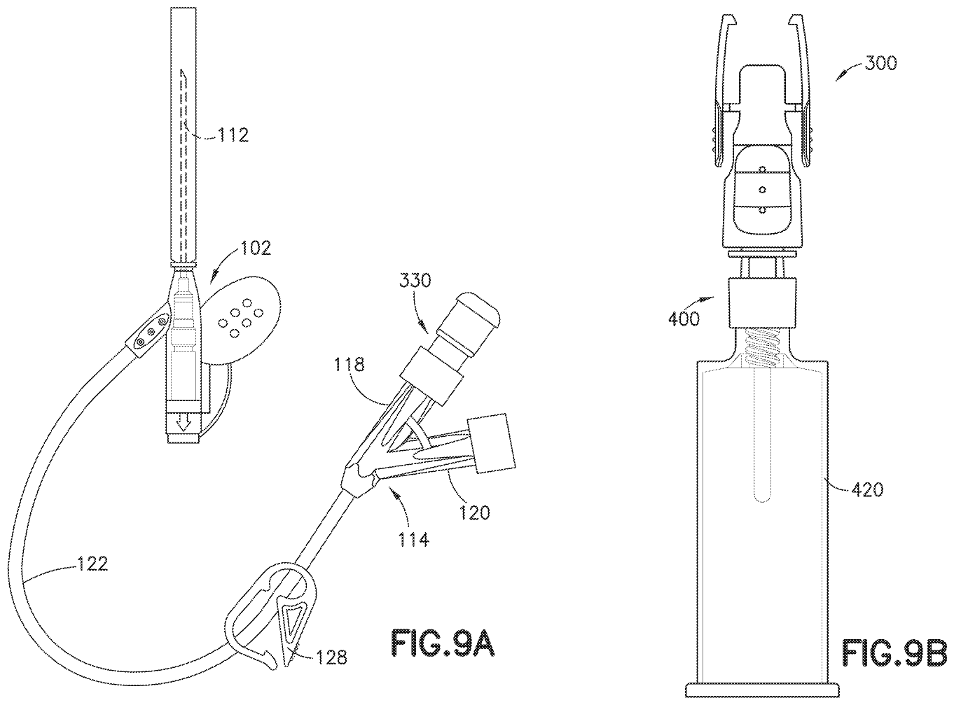

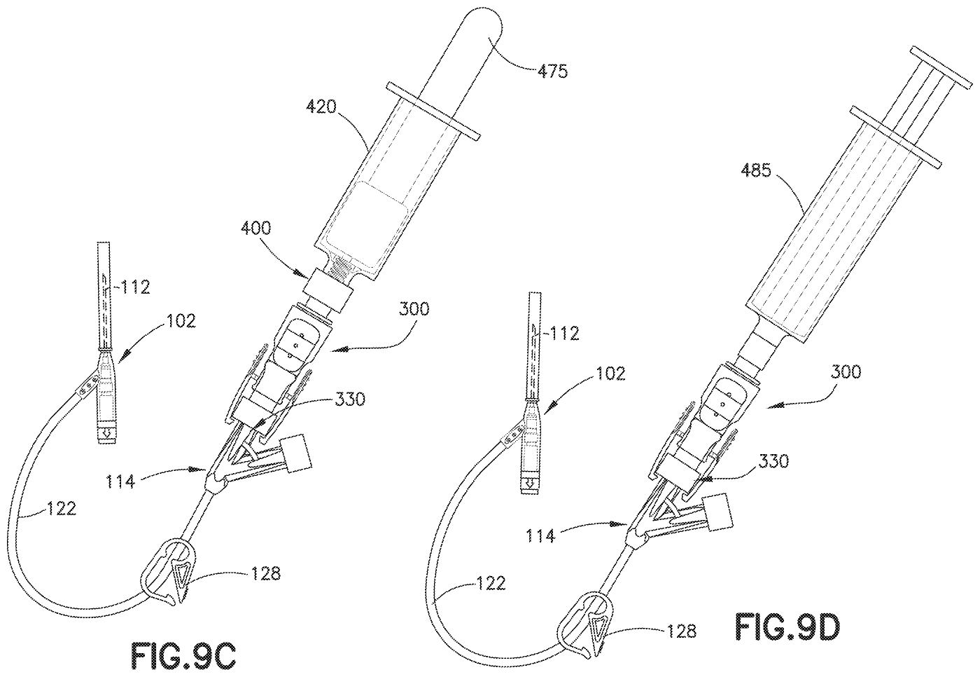

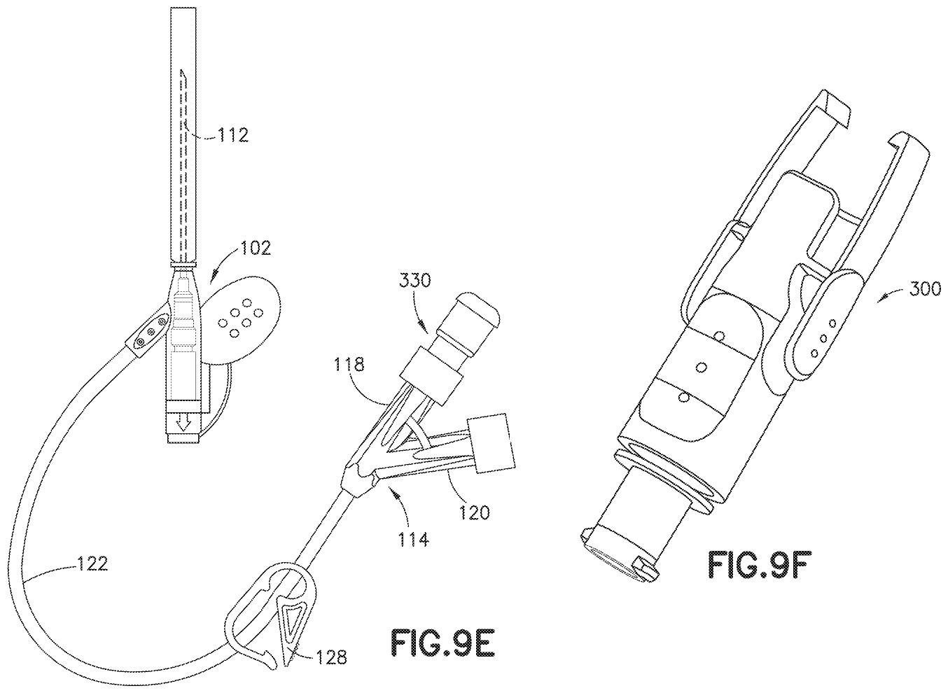

Referring now to FIGS. 9A-9F, the steps of utilizing the catheter system 100 and catheter adapter 102 with the blood collection adapter 300 in accordance with an aspect of the present disclosure are shown. First, referring to FIG. 9A, the catheter adapter 102 is fluidly coupled to a Y-adapter 114, which includes the connector 330 (with septum) coupled thereto. As shown in FIG. 9B and described above with respect to FIGS. 5A and 5B, the blood collection adapter 300 may be coupled to the connector 330 such that a cannula within the blood collection adapter 300 pierces the septum of the connector 330 to place the blood collection adapter 300 in fluid communication with the Y-adapter 114 and, thus, the catheter adapter 102.

Next, referring to FIG. 9C, the luer lock access device 400 may be coupled to the blood collection adapter 300, and a blood collection container 475 may be utilized in conjunction with the luer lock access device 400 in order to draw blood via the catheter adapter 102. As noted above, the blood collection container 475 may be, e.g., a BD VACUTAINER® blood collection tube. Upon completion of the blood draw, the pinch clamp 128 may be closed in order to block the fluid path through the extension tube 122, and the blood collection container 475 and luer lock access device 400 may be decoupled from the blood collection adapter 300. In accordance with another embodiment of the present disclosure, it is to be understood that the blood collection adapter and luer lock access device 400 may be provided pre-attached with the connector 330 and other features of the catheter system 100.

After blood draw and removal of the luer lock access device 400, a syringe 485 may be coupled to the blood collection adapter 300, as is shown in FIG. 9D. As disclosed above, the syringe 485 may contain a flushing fluid such as, e.g., a saline solution. When the plunger of the syringe 485 is depressed, the flushing fluid contained therein may act to flush any residual blood present within the connector 330.

Referring to FIGS. 9E and 9F, upon completion of the flushing step, the syringe 485 may be removed from the blood collection adapter 300, and the blood collection adapter 300 may be decoupled from the connector 330. As is shown in FIG. 9F and discussed in detail above with respect to FIGS. 5A and 5B, the cannula tip of blood collection adapter 300 is substantially protected by the lever arms 346 and/or side members 351 of the blood collection adapter 300 upon removal from the connector 330, thereby helping prevent needle stick injuries. With the blood draw adapter 300 removed after the flushing of residual blood and the pinch clamp 128 opened, the catheter system 100 may be utilized for an infusion, another blood draw, etc.

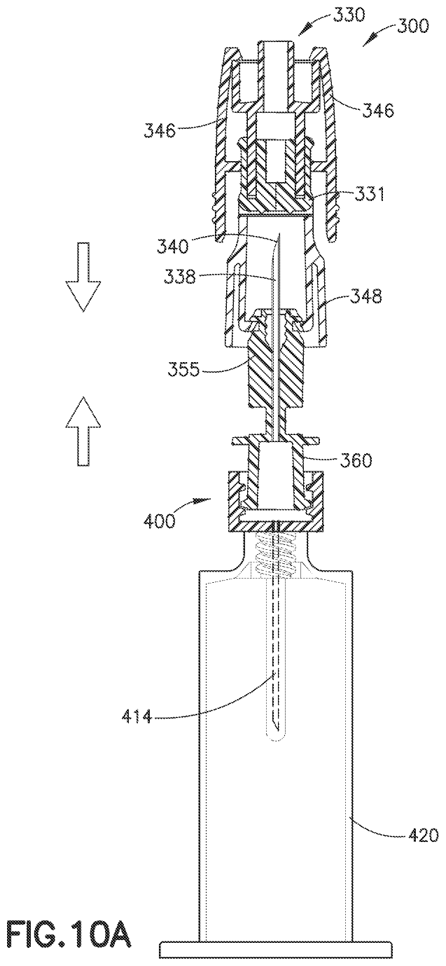

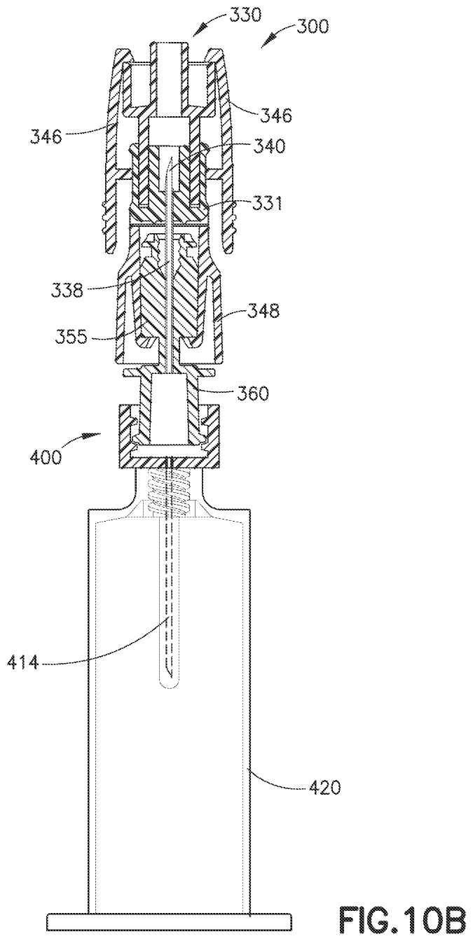

Next, referring to FIGS. 10A and 10B, in some embodiments, the blood collection adapter 300 may require telescopic actuation prior to blood draw. That is, as shown in FIG. 10A, the inner body portion 355 carrying the cannula 338 may initially be positioned proximally (or substantially outside) of the body 348 of the blood collection adapter 300 such that the tip 340 of the cannula 338 is wholly within the body 348 and does not pierce the septum 331. Then, when a blood draw through the blood collection adapter 300 is desired, the inner body portion 355 and/or the body 348 may be actuated relative to one another such that the tip 340 of the cannula 338 pierces the septum 331 and provides fluid communication between the blood collection adapter 300 and the connector 330, as is shown in FIG. 10B. In this way, blood draw into the luer lock access device 400 can be prevented until distal actuation of the inner body portion 355 relative to the body 348.

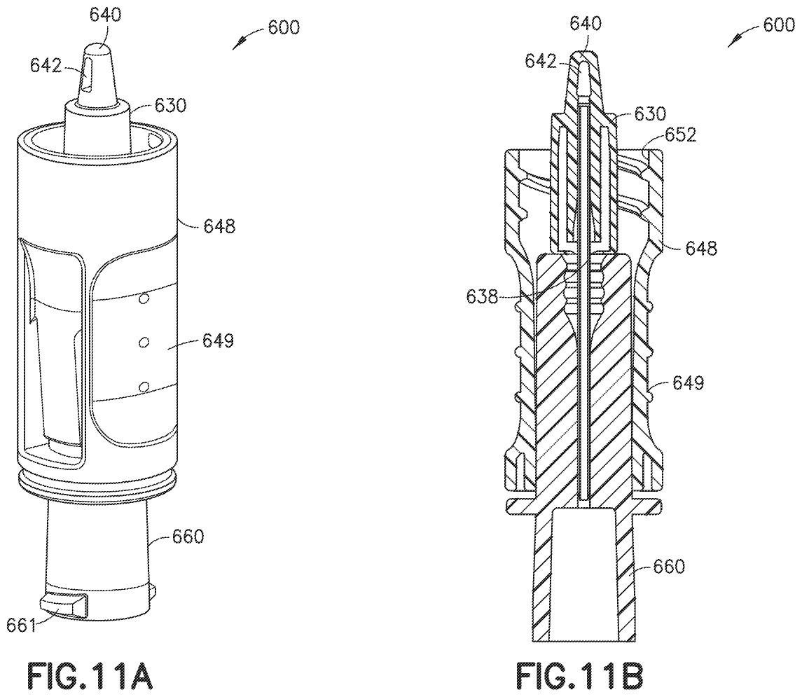

In the embodiments described above with respect to FIGS. 5A-10B, the blood collection adapter 300, 500 is configured to include a cannula having a sharp tip to allow for piercing of, e.g., the septum 331 of connector 300. However, in some alternative embodiments, the connector may be configured as a needleless connector, wherein the septum does not require piercing by a sharp-tipped cannula in order to provide fluid communication between a blood collection adapter and a catheter adapter. Accordingly, referring to FIGS. 11A and 11B, a blood collection adapter 600 in accordance with another aspect of the present disclosure is illustrated. Blood collection adapter 600 is configured for use with a needleless connector and, thus, does not utilize a sharp-tipped cannula, thereby avoiding any potential needle stick injuries related to the blood collection adapter itself.

Blood collection adapter 600 includes a central portion 630 extending from a distal end thereof, with a tip 640 extending distally from the central portion 630. The tip 640 may be a substantially blunt tip capable of use with a needleless connector, as will be described in further detail herein. The tip 640 includes at least one side hole 642. As will be described in further detail below, the at least one side hole 642 may not only accommodate blood draw through the blood collection adapter 600, but may also provide for improved flushing of the needleless connector after blood draw.

Referring still to FIGS. 11A and 11B, the blood collection adapter 600 includes a holder portion 648 having at least one gripping portion 649, as well as an internally threaded portion 652 at least partially surrounding the central portion 630. The internally threaded portion 652 is configured to engage, e.g., the external threads of a needleless connector. Blood collection adapter 600 also includes an adapter hub 660 having a cannula 638 extending therethrough. The adapter hub 660 may include proximal coupling portion 661 configured to secure the adapter hub 660 to, e.g., a luer lock access device. While shown in FIGS. 11A and 11B as a separate component, it is to be understood that, in some embodiments, the adapter hub 660 may be monolithically formed with the blood collection adapter 600.

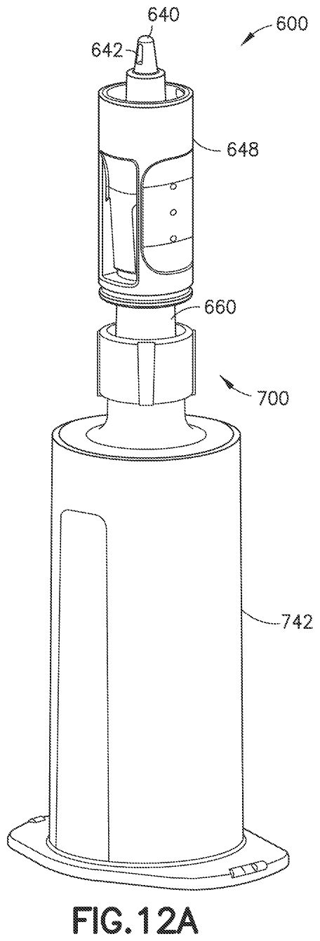

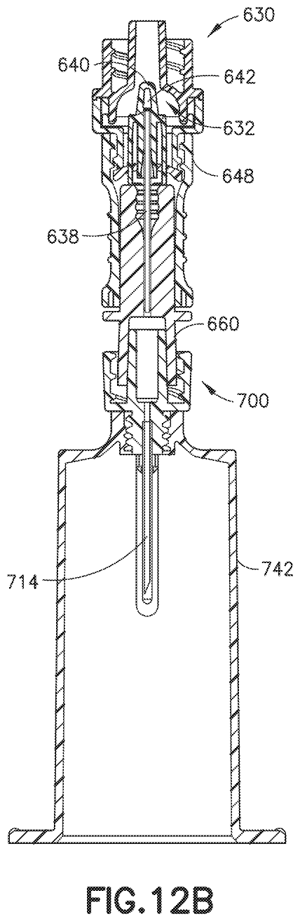

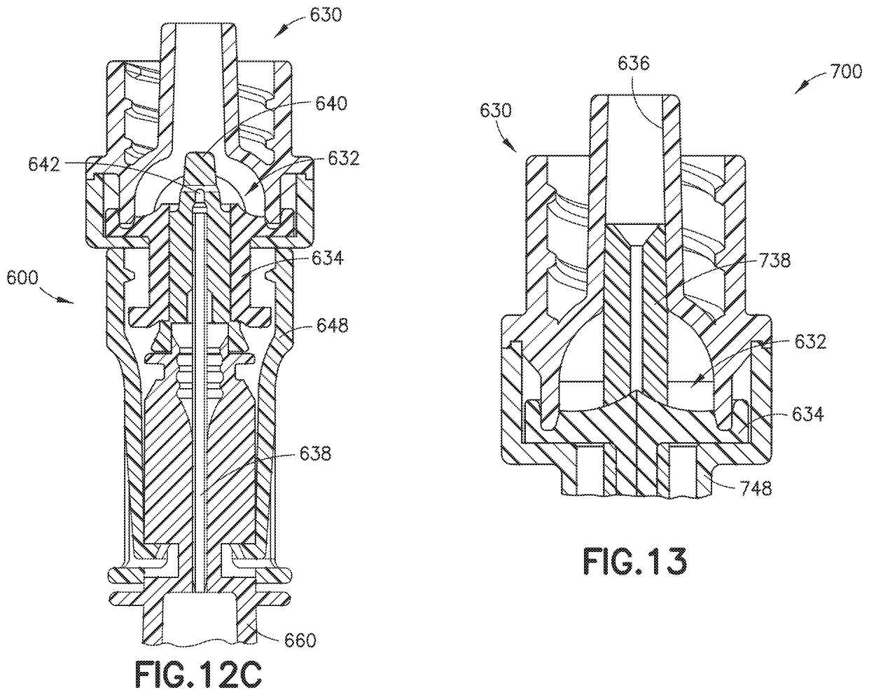

Referring now to FIGS. 12A-12C, in some embodiments, the blood collection adapter 600 may be configured to be coupled to a luer lock access device 700 so as to receive a blood collection device such as, e.g., a BD VACUTAINER® blood collection tube. Similar to luer lock access device 400 described above, the luer lock access device 700 includes a luer lock hub having internal threads capable of engaging the proximal coupling portion 661 of the adapter hub 660 to releasably secure the blood collection adapter 600 to the luer lock access device. The luer lock access device 700 further includes a needle 714 in fluid communication with the luer lock hub so as to allow fluids to pass from the blood collection adapter 600 to the blood collection device. In some embodiments, the needle 714 may be substantially surrounded by a sheath to protect from needle stick injuries. Furthermore, in some embodiments, the luer lock access device 700 may include a holder 742, which is sized and configured to accommodate a blood collection device such as, e.g., a BD VACUTAINER® blood collection tube. However, it is to be understood that other configurations of the luer lock access device, similar to those disclosed above with respect to FIGS. 7A-7C, may be utilized.

Additionally, FIGS. 12B and 12C show that the distal end of blood collection adapter 600 may be coupled to a needleless connector 630, with the tip 640 and at least part of the central portion 630 of the blood collection adapter 600 being configured to pass through a septum 634 of the needleless connector 630. In some embodiments, the septum 634 may be configured to have a slit or other feature so as to allow for the passage of blunt tip 640 and/or central portion 630 therethrough.

Referring again to FIG. 12C, the tip 640 is shown in a fully distal position relative to the connector 630, wherein the tip 640, including at least a portion of the at least one side hole 642, extends through the septum 634 and is in communication with an interior passage 632 of the connector 630. However, the tip 640 is substantially limited in the distance within the interior passage 632 through which it can travel due to the interface between the adapter hub 660 and the body 648, thereby maintaining the tip 640 and the at least one side hole 642 at a position substantially within a proximal portion of the interior passage 632. In this way, not only can the blood collection adapter 600 be used to effectively draw blood through connector 630, but the positioning of the tip 640 and the at least one side hole 642 within the interior passage 632 of connector 630 allows for residual blood to be effectively flushed from the connector 630 after blood draw, as the dead space proximal to the tip 640 within the interior passage 632 is minimized. In some embodiments, at least a portion of the at least one side hole 642 may overlap with the septum 634 so as to ensure that the at least one side hole 642 is below any dead space in the interior passage 632.

While not shown or described in detail, similar to cannula 338 described above, it is to be understood that cannula 638 may be configured to have an internal diameter(s) and length optimized so as to reduce shear stress and, thus, reduce hemolysis during blood draw.

Referring to FIG. 13, a portion of a blood collection adapter 700 in accordance with an aspect of the present disclosure is illustrated. Unlike blood collection adapter 600 described above, which utilizes at least one side hole 642 to flush residual blood from an interior passage of a needleless connector 630, blood collection adapter 700 includes a body 748 having an elongated flush tip 738 extending distally therefrom. The elongated flush tip 738 may be sized and configured so as to extend through the septum 634 and at least partially into a luer tip portion 636 of the needleless connector 630. In this way, the elongated flush tip 738 extends beyond the interior passage 632, thereby bypassing any dead space typically found between the septum top surface and the cannula tip. Thus, blood drawn through the flush tip 738 cannot reach the interior passage 632, obviating the need to flush the interior passage 632 to remove residual blood from the dead space after blood draw.

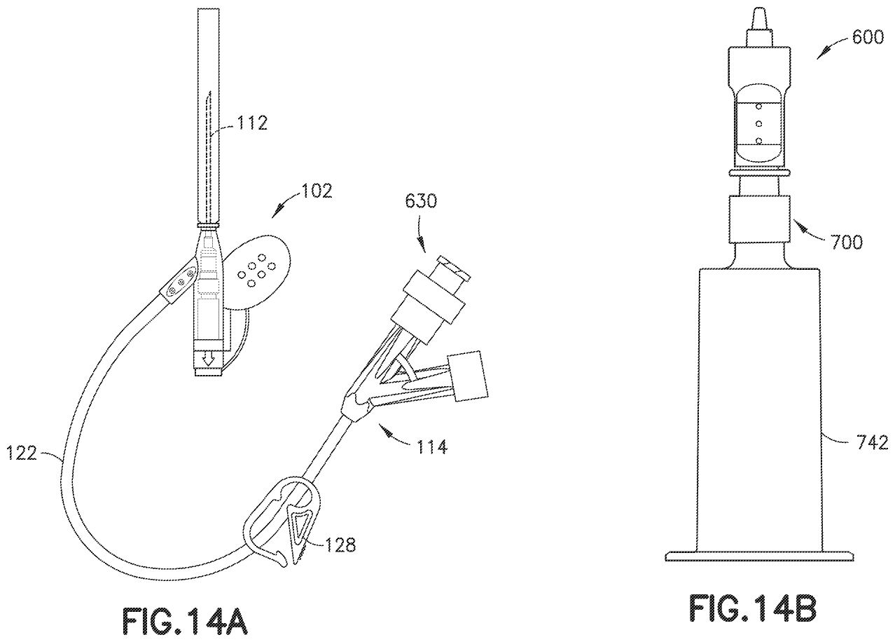

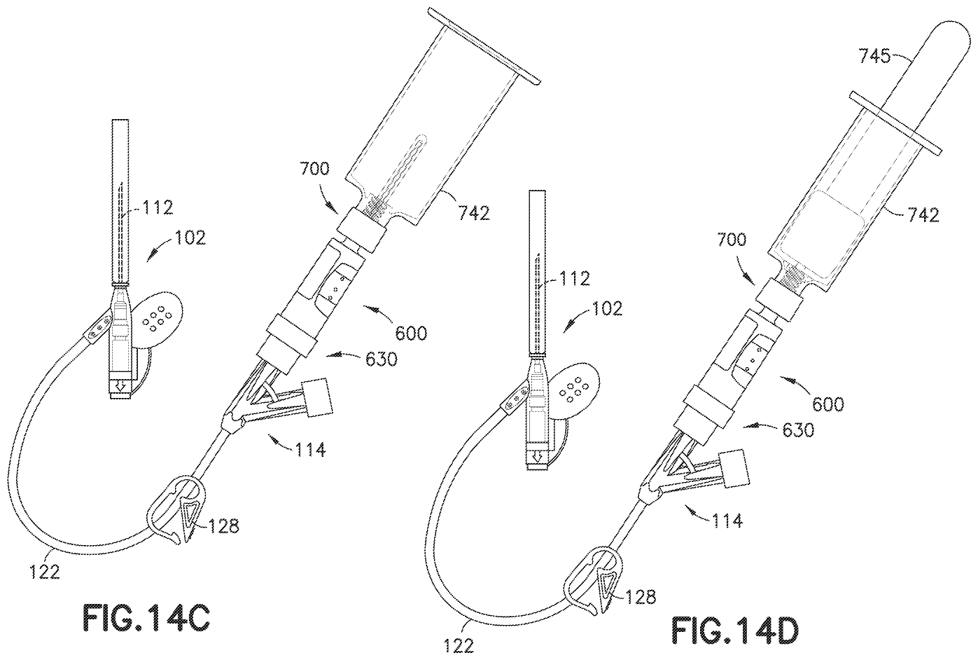

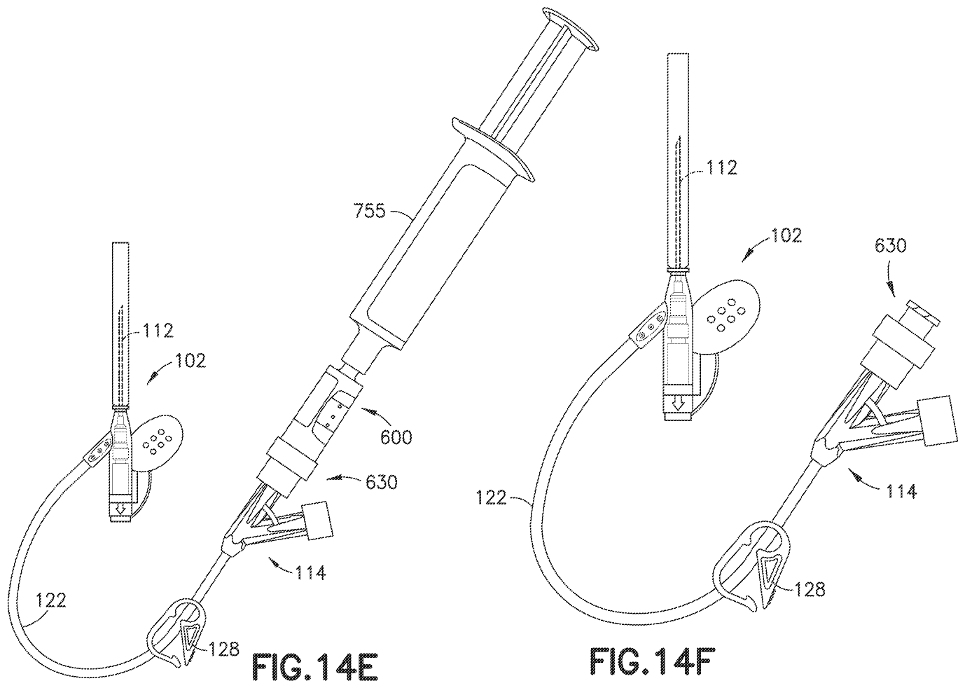

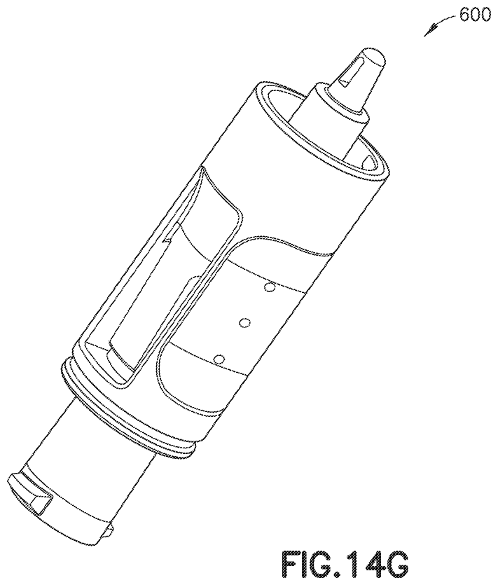

Referring now to FIGS. 14A-14G, the steps of utilizing the catheter system 100 and catheter adapter 102 with the blood collection adapter 600 in accordance with an aspect of the present disclosure are shown. First, referring to FIG. 14A, the catheter adapter 102 is fluidly coupled to a Y-adapter 114, which includes the needleless connector 630 (with septum) coupled thereto. As shown in FIGS. 14B and 14C, and described above with respect to FIGS. 12A-12C, the blood collection adapter 600 may be coupled to the connector 630 such that the tip 640 and the at least one side hole 642 within the blood collection adapter 600 extends through the septum of the connector 630 to place the blood collection adapter 600 in fluid communication with the Y-adapter 114 and, thus, the catheter adapter 102. A luer lock access device 700 with holder 742 may be coupled to the blood collection adapter 600.

Referring to FIG. 14D, a blood collection container 745 may be utilized in conjunction with the luer lock access device 700 in order to draw blood via the catheter adapter 102. As € noted above, the blood collection container 745 may be, e.g., a BD VACUTAINER® blood collection tube. Upon completion of the blood draw, the pinch clamp 128 may be closed in order to block the fluid path through the extension tube 122, and the blood collection container 745 and luer lock access device 700 may be decoupled from the blood collection adapter 600. In accordance with another embodiment of the present disclosure, it is to be understood that the blood collection adapter and luer lock access device 700 may be provided pre-attached with the connector 630 and other features of the catheter system 100.

After blood draw and removal of the luer lock access device 700, a syringe 755 may be coupled to the blood collection adapter 600, as is shown in FIG. 14E. As disclosed above, the syringe 755 may contain a flushing fluid such as, e.g., a saline solution. When the plunger of the syringe 755 is depressed, the flushing fluid contained therein may act to flush any residual blood present within the connector 630.

Referring to FIGS. 14F and 14G, upon completion of the flushing step, the syringe 755 may be removed from the blood collection adapter 600, and the blood collection adapter 600 may be decoupled from the connector 330. As is shown in FIG. 14G and discussed in detail above with respect to FIGS. 11A and 11B, the tip 640 of blood collection adapter 600 is blunt, thereby helping prevent needle stick injuries commonly associated with needle connectors. With the blood draw adapter 600 removed after the flushing of residual blood and the pinch clamp 128 opened, the catheter system 100 may be utilized for an infusion, another blood draw, etc.

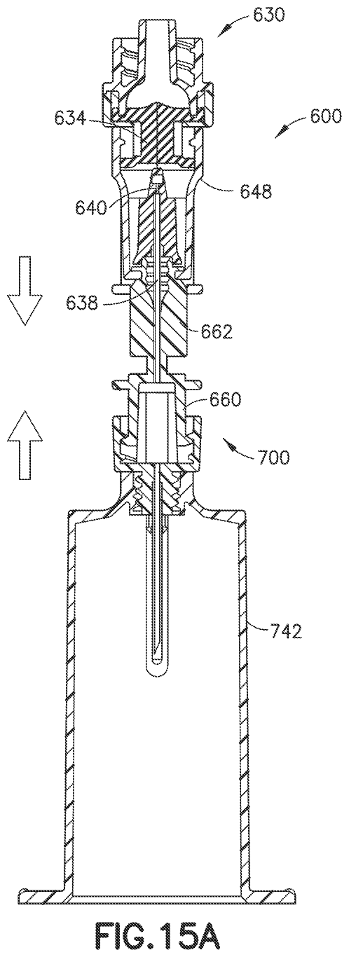

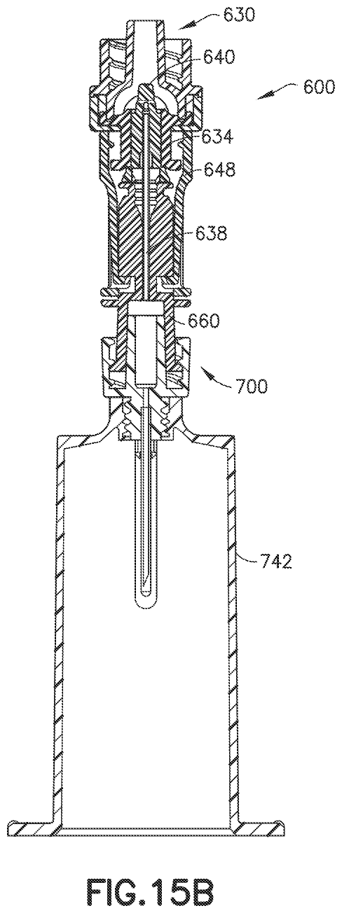

Referring now to FIGS. 15A and 15B, in some embodiments, the blood collection adapter 600 may require telescopic actuation prior to blood draw. That is, as shown in FIG. 15A, an inner body portion 662 carrying the cannula 638 and having the tip 640 positioned distally thereon may initially be at least partially outside of the body 648 of the blood collection adapter 600 such that the tip 640 is wholly within the body 648 and does not extend through the septum 634 of the connector 630. Then, when a blood draw through the blood collection adapter 600 is desired, the inner body portion 662 and/or the body 648 may be actuated relative to one another such that the tip 640 (and the at least one side hole 642, as described above with respect to FIGS. 11A and 11B) extends substantially through the septum 634 and provides fluid communication between the blood collection adapter 600 and the connector 630, as is shown in FIG. 15B. In this way, blood draw into the luer lock access device 700 can be prevented until distal actuation of the inner body portion 662 relative to the body 648.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A catheter system, comprising:
a catheter adapter, comprising a distal end, a proximal end, and a lumen extending through the distal end of the catheter adapter and the proximal end of the catheter adapter;
a catheter extending distally from the distal end of the catheter adapter;
a connector coupled to the catheter adapter and comprising a septum;
a blood collection adapter coupled to the connector, wherein the blood collection adapter is configured to create a fluid path through the connector; and
a blood collection set coupled to the blood collection adapter, the blood collection set comprising a needle assembly configured to receive a blood collection container.

2. The catheter system of claim 1, further comprising:
a side port disposed between the distal end of the catheter adapter and the proximal end of the catheter adapter;
an extension tube, comprising a distal end integrated with the side port of the catheter adapter and a proximal end integrated with the distal end of the adapter; and
an adapter, comprising a distal end and a port, wherein the connector is coupled to the port of the adapter.

3. The catheter system of claim 2, wherein the adapter comprises a Y-adapter.

4. The catheter system of claim 1, wherein the blood collection adapter comprises a distal piece and a proximal piece movable distally towards the distal piece, wherein the proximal piece is configured to activate the septum and create the fluid path through the connector in response to the proximal piece moving distally towards the distal piece.

5. The catheter system of claim 4, wherein the proximal piece of the blood collection adapter comprises a cannula configured to penetrate the septum of the connector to create the fluid path through the connector.

6. The catheter system of claim 5, wherein the needle assembly comprises a body, a sharp needle extending proximally from the body, and a sheath covering a proximal tip of the sharp needle.

7. The catheter system of claim 6, wherein the sharp needle and the cannula are separately formed and have different gauges.

8. The catheter system of claim 6, wherein the sharp needle and the cannula are separately formed and have the same gauge.

9. The catheter system of claim 6, wherein the sharp needle and the cannula are monolithically formed as a single unit.

10. The catheter system of claim 9, wherein the blood collection adapter and the body of the needle assembly are monolithically formed as a single unit.

11. The catheter system of claim 4, wherein the proximal piece of the blood collection adapter comprises a male luer, wherein the connector comprises a needleless connector.

12. The catheter system of claim 11, wherein a distal end of the cannula comprises a chamfer or a flow diverter.

13. The catheter system of claim 1, wherein the blood collection adapter and the body of the needle assembly are monolithically formed as a single unit.

14. The catheter system of claim 1, further comprising an extension set, wherein the extension set comprises a distal end coupled to the proximal end of the catheter adapter and a proximal end coupled to the connector.

15. A blood collection assembly to couple to a catheter assembly, the blood collection assembly comprising:
a needle assembly, comprising a body, a sharp needle extending proximally from the body, and a sheath covering a proximal tip of the sharp needle; and
a blood collection adapter coupled to a distal end of the needle assembly, wherein the blood collection adapter is configured to create a fluid path through a connector.

16. The blood collection assembly of claim 15, wherein the blood collection adapter is directly and removably coupled to the needle assembly.

17. The blood collection assembly of claim 15, wherein the blood collection adapter is monolithically formed as a single unit with the body of the needle assembly.

18. The blood collection assembly of claim 15, wherein the blood collection adapter comprises a distal piece and a proximal piece movable distally towards the distal piece, wherein the proximal piece of the blood collection adapter comprises a male luer or a cannula, wherein the male luer or the cannula is configured to activate a septum of the connector and create the fluid path through the connector in response to the proximal piece moving distally towards the distal piece.

19. A method, comprising:
coupling a blood collection assembly to a catheter assembly, wherein the catheter assembly comprises:
a catheter adapter, comprising a distal end, a proximal end, and a lumen extending through the distal end of the catheter adapter and the proximal end of the catheter adapter;
a catheter extending distally from the distal end of the catheter adapter;
and a connector coupled to the catheter adapter and comprising a septum;
wherein the blood collection assembly comprises a needle assembly and a blood collection adapter, wherein the blood collection adapter comprises a distal piece and a proximal piece movable distally towards the distal piece, wherein the proximal piece of the blood collection adapter comprises a male luer or a cannula;
moving the proximal piece distally towards the distal piece, wherein the male luer or the cannula is configured to activate the septum of the connector and create a fluid path through the connector in response to moving the proximal piece distally towards the distal piece; and
collecting blood in a blood collection device coupled to the needle assembly after moving the proximal piece distally towards the distal piece.

20. The method of claim 19, wherein the catheter assembly further comprises:
a side port disposed between the distal end of the catheter adapter and the proximal end of the catheter adapter;
an extension tube, comprising a distal end integrated with the side port of the catheter adapter and a proximal end integrated with the distal end of the adapter; and
an adapter, comprising a distal end and a port, wherein the connector is coupled to the port of the adapter.

21.-40. (canceled)
